(19) United States
(12) Patent Application Publication
(10) Pub. No.: US 2006/0264433 A1
(43) Pub. Date: Nov. 23, 2006

(54) Pharmaceutical compositions as inhibitors of dipeptidyl peptidase-IV (DPP-IV)

(76) Inventors: Bradley J. Backes, Chicago, IL (US); Gregory L. Hamilton, Chicago, IL (US); Hana A. Kopecka, Vernon Hills, IL (US); Chunqiu Lai, Libertyville, IL (US); Kenton L. Longenecker, Grayslake, IL (US); David J. Madar, Gurnee, IL (US); Zhonghua Pei, Libertyville, IL (US); Kent D. Stewart, Gurnee, IL (US); Thomas W. Von Geldern, Richmond, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/438,881

(22) Filed: May 23, 2006

Related U.S. Application Data
(60) Provisional application No. 60/683,743, filed on May 23, 2005.

(51) Int. Cl. A61K 31/5377; A61K 31/506; A61K 31/502; A61K 31/496; A61K 31/53; A61K 31/4439; A61K 31/4025; C07D 413/02; C07D 409/02; C07D 403/02

(52) U.S. Cl. 514/235.2; 514/248; 514/241; 514/254.01; 514/256; 514/406; 514/422; 514/343; 544/141; 544/237; 544/333; 544/363; 544/209; 546/277.1; 548/527

(57) ABSTRACT

The present invention relates to compounds, which inhibit dipeptidyl peptidase IV (DPP-IV) and are useful for the prevention or treatment of diabetes, especially type II diabetes, as well as hyperglycemia, syndrome X, hyperinsulinemia, obesity, atherosclerosis, various immunomodulatory diseases, and other diseases.

### CROSS-REFERENCE SECTION TO RELATED APPLICATIONS

This application claims the benefit of priority of U.S. Provisional Application Ser. No. 60/683,743, which was filed May 23, 2005, and is incorporated herein by reference in its entirety.

### FIELD OF THE INVENTION

The present invention relates to compounds, which inhibit dipeptidyl peptidase IV (DPP-IV) and are useful for the prevention or treatment of diabetes, especially type II diabetes, as well as hyperglycemia, syndrome X, hyperinsulinemia, obesity, atherosclerosis, various immunomodulatory diseases, gastrointestinal diseases and disorders, cancer, cardiovascular diseases, cerebrovascular diseases, anxiety, depression, insomnia, cognitive disorders, diseases and disorders of the central nervous system, inflammation and inflammatory diseases, respiratory diseases and disorders, musculoskeletal disorders, osteoporosis, and menopausal symptoms and disorders.

### BACKGROUND OF THE INVENTION

Dipeptidyl peptidase IV (DPP-IV, CD26, EC 3.4.14.5) is a serine protease with specificity for cleaving Xaa-Pro and, to a lesser extent, Xaa-Ala dipeptides from the N-termini of polypeptides and proteins. DPP-IV is a non-classical serine protease in that the catalytic triad of Ser-Asp-His, found in the C-terminal region of the enzyme, is in reverse order to that found in classical serine proteases. DPP-IV is widely expressed in mammalian tissue as a type II integral membrane protein. DPP-IV is expressed on the surface of differentiated epithelial cells of the intestine, liver, kidney proximal tubules, prostate, corpus luteum, and on leukocyte subsets such as lymphocytes and macrophages. A soluble form of the enzyme is found in serum that has structure and function identical to the membrane-bound form of the enzyme but lacks the hydrophobic transmembrane domain.

DPP-IV has many physiologically relevant substrates such as chemokines, RANTES (regulated on activation normal T cell expressed and secreted), eotaxin, and macrophage-derived chemokine, neuropeptides such as NPY (neuropeptide Y) and substance P, vasoactive peptides, and incretins such as GLP-1 (glucagon-like peptide-1) and GIP (gastric inhibitory peptide/glucose-dependent insulinotropic polypeptide). GLP-1 is a 30 amino acid peptide hormone produced in the L cells of the distal small intestine in response to ingested nutrients. GLP-1 binding to its receptor on various tissues stimulates insulin gene expression, biosynthesis and glucose-dependent insulin secretion, inhibits glucagon secretion, promotes satiety, slows gastric emptying and promotes growth of pancreatic beta cells. Based on this profile, GLP-1 -based therapies are expected to be beneficial in the treatment of type II diabetes and obesity. Studies in which type II diabetic patients have been infused with GLP-1 have demonstrated efficacy in normalizing both fasted and prandial glycemia. However, active GLP-1 (7-36) amide is rapidly converted by DPP-IV to GLP-1 (9-36), which is inactive or is a receptor antagonist. The short half-life of GLP-1 in the circulation (1-1.5 minutes) is a major obstacle to its use as a therapeutic agent. To circumvent the drawback of the short half-life of GLP-1, inhibitors of DPP-IV, the primary degradative enzyme of GLP-1, increase the level of active circulating GLP-1 (7-36) amide. DPP-IV inhibitors have been demonstrated to improve glucose tolerance in type II diabetes.

The inhibition of DPP-IV provides for an attractive therapeutic treatment for type II diabetes and obesity. Although DPP-IV inhibitors have demonstrated improved glucose tolerance in type II diabetes, many suffer from having short half-life and toxicity. Therefore, there is a need for DPP-IV inhibitors having an improved pharmacological profile as an alternative for the treatment of type II diabetes.

### SUMMARY OF THE INVENTION

The present invention is directed to compounds of formula (I), 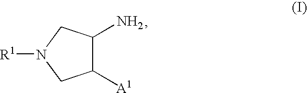
or therapeutically acceptable salts, prodrugs, salts of prodrugs, or metabolites thereof, wherein

A¹ is aryl or heteroaryl;

R¹ is R², R²C(O)—, R²OC(O)—, R²(CH₂)ₙC(O)—, R²(CH₂)ₙOC(O)—, R²NHC(O)—, R²(CH₂)ₙNHC(O)—, (R²)(R^{1A})NC(O)—, or R²(CH₂)ₙN(R^{1A})C(O)—;

n is 1, 2, 3, 4, 5 or 6;

R^{1A} is alkyl;

R² is aryl, heteroaryl or heterocyclyl, each of which is unsubstituted or substituted with one or two or three substituents independently selected from the group consisting of R⁴, R⁴O—, R⁴ S—, R⁴S(O)—, R⁴SO₂—, R⁴C(O)—, R⁴OC(O)—, H₂N—, (R⁴)(H)N—, (R⁴)₂N—, NH₂C(O)—, (R⁴)(H)NC(O)—, (R⁴)₂NC(O)—, H₂NSO₂—, (R⁴)(H)NSO₂—, (R⁴)₂NSO₂—, R⁴C(O)NH—, R⁴C(O)NH—, R⁴C(O)NR⁵—, —CN, —OH, —NO₂, —CHO, —CO₂H, —CF₃, —CF₂CF₃, —OCF₃, —OCF₂CF₃, F, Cl, Br and I;

R⁴ is R⁵ or R^{6;}

R⁵ is cycloalkyl, cycloalkenyl, aryl, heteroaryl or heterocyclyl;

R⁶ is alkyl which is unsubstituted or substituted with one or two or three substituents independently selected from the group consisting of R⁷, R⁷O—, R⁷S—, R7S(O)—, R⁷SO₂—, R⁷C(O)—, R⁷OC(O)—, H₂N—, (R⁷)(H)N—, (R⁷)₂N—, NH₂C(O)—, (R⁷)(H)NC(O)—, (R⁷)₂NC(O)—, H₂NSO₂—, (R⁷)(H)NSO₂—, (R⁷)₂NSO₂—, R⁷C(O)NH—, R⁷C(O)NR⁷—, —CN, —OH, —CHO, —CO₂H, F, Cl, Br and I;

R⁷ is alkyl, aryl, heteroaryl or heterocyclyl;

wherein the cycloalkyl, cycloalkenyl, aryl, heteroaryl and heterocyclyl moieties represented by A¹, R⁵ and R⁷ are independently unsubstituted or substituted with one or two or three or four or five substituents independently selected from the group consisting of R⁸, R⁸O—, R⁸S—, R⁸S(O)—, R⁸SO₂—, R⁸C(O)—, R⁸OC(O)—, H₂N—, (R⁸)(H)N—, (R⁸)₂N—, NH₂C(O)—, (R⁸)(H)NC(O)—, (R⁸)₂NC(O)—, H₂NSO₂—, (R⁸)(H)NSO₂—, (R⁸)₂NSO₂—, R⁸C(O)NH—, R⁸C(O)NR⁸—, O═, —CN, —OH, —NO₂, —CHO, —CO₂H, —CF₃, —CF₂CF₃, —OCF₃, —OCF₂CF₃, F, Cl, Br and I;

R⁸ is R⁹ or R¹¹;

R⁹ is cycloalkyl, cycloalkenyl, aryl, heteroaryl or heterocyclyl, each of which is unsubstituted or substituted with one or two or three or four substituents independently selected from the group consisting of R¹⁰, R¹⁰O—, R¹⁰S—, R¹⁰S(O)—, R¹⁰SO₂—, R¹⁰OC(O)—, R¹⁰OC(O)—, H₂N—, (R¹⁰)(H)N—, (R¹⁰)₂N—, NH₂C(O)—, (R¹⁰)(H)NC(O)—, (R¹⁰)₂NC(O)—, H₂NSO₂—, (R¹⁰)(H)NSO₂—, (R¹⁰)₂NSO₂—, R¹⁰C(O)NH—, R¹⁰C(O)NR¹⁰—, O═, —CN, —OH, —NO₂, —CHO, —CO₂H, —CF₃, —CF₂CF₃, —OCF₃, —OCF₂CF₃, F, Cl, Br and I;

R¹⁰ is alkyl, which is unsubstituted or substituted with one substituent selected from the group consisting of —OR¹², R¹²S—, R¹²S(O)—, R¹²SO₂—, aryl, heteroaryl and heterocyclyl;

R¹¹ is alkyl which is unsubstituted or substituted with one substituent selected from the group consisting of —OR¹², R¹²S—, R¹²S(O)—, R¹²SO₂—, aryl, heteroaryl and heterocyclyl; and

R¹² is alkyl.

Additionally, the present invention provides methods of treating various diseases with the compounds of the present invention. Furthermore, the present invention provides a pharmaceutical composition.

### DETAILED DESCRIPTION OF THE INVENTION

Variable moieties of compounds herein are represented by identifiers (capital letters with numerical and/or alphabetical superscripts) and can be specifically embodied.

It is meant to be understood that proper valences are maintained for all moieties and combinations thereof and the point of attachment of monovalent moieties having more than one atom is shown by the symbol “-”.

It is also meant to be understood that a specific embodiment of a variable moiety can be the same or different as another specific embodiment having the same identifier.

The term “alkyl,” as used herein, means, but is not limited to, C₁-alkyl, C₂-alkyl, C₃-alkyl, C₄-alkyl, C₅-alkyl and C₆-alkyl.

The term “C₁-alkyl,” as used herein, means, but is not limited to, methyl.

The term “C₂-alkyl,” as used herein, means, but is not limited to, ethyl.

The term “C₃-alkyl,” as used herein, means, but is not limited to, prop-1-yl and prop-2-yl(isopropyl).

The term “C₄-alkyl,” as used herein, means, but is not limited to, but-1-yl, but-2-yl, 2-methylprop-1-yl and 2-methylprop-2-yl(tert-butyl).

The term “C₅-alkyl,” as used herein, means, but is not limited to, 2,2-dimethylprop-1-yl(neo-pentyl), 2-methylbut-1-yl, 2-methylbut-2-yl, 3-methylbut-1-yl, 3-methylbut-2-yl, pent-1-yl, pent-2-yl and pent-3-yl.

The term “C₆-alkyl,” as used herein, means, but is not limited to, 2,2-dimethylbut-1-yl, 2,3-dimethylbut-1-yl, 2,3-dimethylbut-2-yl, 3,3-dimethylbut-1-yl, 3,3-dimethylbut-2-yl, 2ethylbut-1-yl, hex-1-yl, hex-2-yl, hex-3-yl, 2-methylpent-1-yl, 2-methylpent-2-yl, 2-methylpent 3-yl, 3-methylpent-1-yl, 3-methylpent-2-yl, 3-methylpent-3-yl, 4-methylpent-1-yl and 4-methylpent-2-yl.

The term “aryl” as used herein, means, but is not limited to, phenyl which is unfused or fused with distal benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or A¹. A¹ is cycloalkane or cycloalkene, each having one or two or three CH₂ moieties unreplaced or replaced with independently selected O, S, S(O), S₂ or NH and one or two CH moieties unreplaced or replaced with N. The distal rings are also unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or A¹.

The term “cycloalkane,” as used herein, means, but is not limited to, C₃-cycloalkane (cyclopropane), C₄-cycloalkane (cyclobutane), C₅-cycloalkane (cyclopentane) and C₆-cycloalkane (cyclohexane).

The term “cycloalkene,” as used herein, means, but is not limited to, C₄-cycloalkene, C₅-cycloalkene and C₆-cycloalkene.

The term “C₄-cycloalkene,” as used herein, means, but is not limited to, cyclobutene and 1,3-cyclobutadiene.

The term “C₅-cycloalkene,” as used herein, means, but is not limited to, cyclopentene and 1,3 -cyclopentadiene.

The term “C₆-cycloalkene,” as used herein, means, but is not limited to, cyclohexene, 1,3-cyclohexadiene and 1,4-cyclohexadiene.

The term “cycloalkenyl,” as used herein, means, but is not limited to, C₃-cycloalkenyl, C₄-cycloalkenyl, C₅-cycloalkenyl and C₆-cycloalkenyl.

The term “C₃-cycloalkenyl,” as used herein, means, but is not limited to, cycloprop-1-en-1-yl and cycloprop-2-en-1-yl.

The term “C₄-cycloalkenyl,” as used herein, means, but is not limited to, cyclobut-1-en-1-yl and cyclobut-2-en-1-yl.

The term “C₅-cycloalkenyl,” as used herein, means, but is not limited to, cyclopent-1-en-1-yl, cyclopent-2-en-1-yl, cyclopent-3-en-1-yl and cyclopenta-1,3-dien-1-yl.

The term “C₆-cycloalkenyl,” as used herein, means, but is not limited to, cyclohex-1-en-1-yl, cyclohex-2-en-1-yl, cyclohex-3-en-1-yl, cyclohexa-1,3-dien-1-yl, cyclohexa-1,4-dien-1-yl, cyclohexa-1,5-dien-1-yl, cyclohexa-2,4-dien-1-yl and cyclohexa-2,5-dien-1-yl.

The term “cycloalkyl,” as used herein, means, but is not limited to, C₃-cycloalkyl (cycloprop-1-yl), C₄-cycloalkyl (cyclobut-1-yl), C₅-cycloalkyl (cyclopent-1-yl) and C₆-cycloalkyl (cyclohex-1-yl).

The term “heteroaryl” as used herein, means, but is not limited to, furanyl, imidazolyl, isothiazolyl, isoxazolyl, 1,2,3-oxadiazoyl, 1,2,5-oxadiazolyl, oxazolyl, pyrazinyl, pyrazolyl, pytidazinyl, pyridinyl, pyrimidinyl, pyrrolyl, tetrazolyl, thiazolyl, thiophenyl, triazinyl and 1,2,3-triazolyl, each of which is unfused or fused with distal benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or B¹. B¹ is cycloalkane or cycloalkene, each having one or two or three CH₂ moieties unreplaced or replaced with independently selected O, S, S(O), SO₂ or NH and one or two CH moieties unreplaced or replaced with N. The distal rings are also unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or B¹.

The term “heterocyclyl,” as used herein, means, but is not limited to, heterocycloalkyl and heterocycloalkenyl.

The term “heterocycloalkenyl,” as used herein, means, but is not limited to, cycloalkenyl having one or two or three CH₂ moieties replaced with independently selected O, S, S(O), SO₂ or NH and one or two CH moieties unreplaced or replaced with N and also means cycloalkene having one or two or three CH₂ moieties unreplaced or replaced with independently selected 0, S, S(O), SO₂ or NH and one or two CH moieties replaced with N, each of which is unfused or fused with distal benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or C¹. C¹ is cycloalkane or cycloalkene, each having one or two or three CH₂ moieties unreplaced or replaced with independently selected O, S, S(O), SO₂ or NH and one or two CH moieties unreplaced or replaced with N. The distal rings fused are also unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or C¹.

The term “heterocycloalkenyl,” as used herein, means, but is not limited to, cycloalkyl having one or two or three CH₂ moieties replaced with independently selected O, S, S(O), SO₂ or NH and one or two CH₂ moieties unreplaced or replaced with N and also means cycloalkane having one or two or three CH₂ moieties unreplaced or replaced with independently selected O, S, S(O), SO₂ or NH and one or two CH moieties replaced with N, each of which is unfused or fused with distal benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or D¹. D¹ is cycloalkane or cycloalkene, each having one or two or three CH₂ moieties unreplaced or replaced with independently selected O, S, S(O), SO₂ or NH and one or two CH moieties unreplaced or replaced with N. The distal rings are also unfused or fused with benzene, furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine, 1,2,3-triazole or D¹.

In an embodiment of the present invention, there is provided compounds of formula (I), 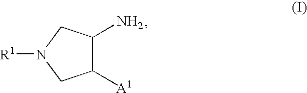
or therapeutically acceptable salts, prodrugs, salts of prodrugs, or metabolites thereof, wherein

A¹ is aryl or heteroaryl;

R¹ is R², R²C(O)—, R²OC(O)—, R²(CH₂)ₙC(O)—, R²(CH₂)ₙOC(O)—, R²NHC(O)—, R²(CH₂)ₙNHC(O)—, (R²)(R^{1A})NC(O)—, or R²(CH₂)ₙN(R^{1A})C(O)—;

n is 1, 2, 3, 4, 5 or 6;

R¹ is alkyl;

R² is aryl, heteroaryl or heterocyclyl, each of which is unsubstituted or substituted with one or two or three substituents independently selected from the group consisting of R⁴, R⁴O—, R⁴S—, R⁴S(O)—, R⁴SO₂—, R⁴C(O)—, R⁴OC(O)—, H₂N—, (R⁴)(H)N—, (R⁴)₂N—, NH₂C(O)—, (R⁴)(H)NC(O)—, (R⁴)₂NC(O)—, H₂NSO₂—, (R⁴)(H)NSO₂—, (R⁴)₂NSO₂—, R⁴C(O)NH—, R⁴C(O)NR⁵—, —CN, —OH, —NO₂, —CHO, —CO₂H, —CF₃, —CF₂CF₃, —OCF₃, —OCF₂CF₃, F, Cl, Br and I;

R⁴ is R⁵ or R⁶;

R⁵ is cycloalkyl, cycloalkenyl, aryl, heteroaryl or heterocyclyl;

R⁶ is alkyl which is unsubstituted or substituted with one or two or three substituents independently selected from the group consisting of R⁷, R⁷O—, R⁷S—, R⁷S(O)—, R⁷SO₂—, R⁷C(O)—, R⁷OC(O)—, H₂N—, (R⁷)(H)N—, (R⁷)₂N—, NH₂C(O)—, (R⁷)(H)NC(O)—, (R⁷)₂NC(O)—, H₂NSO₂—, (R⁷)(H)NSO₂—, (R⁷)₂NSO₂—, R⁷C(O)NH—, R⁷C(O)NR⁷—, —CN, —OH, —CHO, —CO₂H, F, Cl, Br and I;

R⁷ is alkyl, aryl, heteroaryl or heterocyclyl;

wherein the cycloalkyl, cycloalkenyl, aryl, heteroaryl and heterocyclyl moieties represented by A¹, R⁵ and R⁷ are independently unsubstituted or substituted with one or two or three or four or five substituents independently selected from the group consisting of R⁸, R⁸O—, R⁸S—, R⁸S(O)—, R⁸SO₂—, R⁸C(O)—, R⁸OC(O)—, H₂N—, (R⁸)(H)N—, (R⁸)₂N—, NH₂C(O)—, (R⁸)(H)NC(O)—, (R⁸)₂NC(O)—, H₂NSO₂—, (R⁸)(H)NSO₂—, (R⁸)₂NSO₂—, R⁸C(O)NH—, R⁸C(O)NR⁸—, O═, —CN, —OH, —NO₂, —CHO, —CO₂H, —CF₃, —CF₂CF₃, —OCF₃, —OCF₂CF₃, F, Cl, Br and I;

R⁸ is R⁹ or R¹¹;

R⁹ is cycloalkyl, cycloalkenyl, aryl, heteroaryl or heterocyclyl, each of which is unsubstituted or substituted with one or two or three or four substituents independently selected from the group consisting of R¹⁰, R¹⁰O—, R¹⁰S—, R¹⁰S(O)—, R¹⁰SO₂—, R¹⁰C(O)—, R¹⁰OC(O)—, H₂N—, (R¹⁰)(H)N—, (R¹⁰)₂N—, NH₂C(O)—, (R¹⁰)(H)NC(O)—, (R¹⁰)₂NC(O)—, H₂NSO₂—, (R¹⁰)(H)NSO₂—, (R¹⁰)₂NSO₂—, R¹⁰C(O)NH—, R¹⁰C(O)NR¹⁰—, O═, —CN, —OH, —NO₂, —CHO, —CO₂H, —CF₃, —CF₂CF₃, —OCF₃, —OCF₂CF₃, F, Cl, Br and I;

R¹⁰ is alkyl, which is unsubstituted or substituted with one substituent selected from the group consisting of —OR¹², R¹²S—, R¹²S(O)—, R¹²SO₂—, aryl, heteroaryl and heterocyclyl;

R¹¹ is alkyl which is unsubstituted or substituted with one substituent selected from the group consisting of —OR¹², R¹²S—, R¹²S(O)—, R¹²SO₂—, aryl, heteroaryl and heterocyclyl; and

R¹² is alkyl.

Another embodiment pertains to compounds having formula (I), and therapeutically acceptable salts, prodrugs, salt of a prodrugs and metabolites thereof, wherein

A¹ is aryl or heteroaryl;

R¹ is R², R²C(O)—, R²OC(O)—, R²(CH₂)ₙC(O)—, R²(CH₂)ₙOC(O)—, R²NHC(O)—, R²(CH₂)ₙNHC(O)—, (R²)(R^{1A})NC(O)—, or R²(CH₂)ₙN(R^{1A})C(O)—;

n is 1, 2, 3, 4, 5 or 6;

R^{1A} is alkyl;

R² is aryl, heteroaryl or heterocyclyl, each of which is unsubstituted or substituted with one or two or three substituents independently selected from the group consisting of R⁴, R⁴O—, R⁴S—, R⁴S(O)—, R⁴SO₂—, R⁴C(O)—, R⁴OC(O)—, H₂N—, (R⁴)(H)N—, (R⁴)₂N—, NH₂C(O)—, (R⁴)(H)NC(O)—, (R⁴)₂NC(O)—, H₂NSO₂—, (R⁴)(H)NSO₂—, (R⁴)₂NSO₂—, R⁴C(O)NH—, R⁴C(O)NR⁵—, —CN, —OH, —NO₂, —CHO, —CO₂H, —CF₃, —CF₂CF₃, —OCF₃, —OCF₂CF₃, F, Cl, Br and I;

R⁴ is R⁵ or R⁶;

R⁵ is cycloalkyl, cycloalkenyl, aryl, heteroaryl or heterocyclyl;

R⁶ is alkyl which is unsubstituted or substituted with one or two or three substituents independently selected from the group consisting of R⁷, R⁷O—, R⁷S—, R⁷S(O)—, R⁷SO₂—, R⁷C(O)—, R⁷OC(O)—, H₂N—, (R⁷)(H)N—, (R⁷)₂N—, NH₂C(O)—, (R⁷)(H)NC(O)—, (R⁷)₂NC(O)—, H₂NSO₂—, (R⁷)(H)NSO₂—, (R⁷)₂NSO₂—, R⁷C(O)NH—, R⁷C(O)NR⁷—, —CN, —OH, —CHO, —CO₂H, F, Cl, Br and I; and

R⁷ is alkyl, aryl, heteroaryl or heterocyclyl;

wherein the moieties represented by A¹ are substituted with one or two or three substituents independently selected from the group consisting of F, Cl, and O═;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —C₂H and F;

the moieties represented by R⁷ are unsubstituted with one or two independently selected substituents independently selected from the group consisting of alkyl, F, and O═;

R⁸ is R⁹ or R¹¹;

R⁹ is cycloalkyl, heteroaryl or heterocyclyl, each of which is unsubstituted or substituted with one substituent selected from the group consisting of R¹⁰, R¹⁰SO₂—, R¹⁰C(O)—, and O═;

R¹⁰ is alkyl which is unsubstituted or substituted with one substituent selected from the group consisting of —OR¹², R¹²S—, R¹²S(O)—, R¹²SO₂—, aryl, heteroaryl and heterocyclyl; and

R¹¹ is alkyl, which is unsubstituted or substituted with heterocyclyl.

Still another embodiment pertains to compounds having formula (I) or therapeutically acceptable salts, prodrugs, salts of prodrugs, or metabolites thereof, wherein:

A¹ is aryl or heteroaryl;

R¹ is R², R²(CH₂)ₙNHC(O)—, R²(CH₂)ₙN(R^{1A})C(O)— or R²(CH₂)ₙC(O)—;

n is 1, 2, 3, 4, 5 or 6;

R^{1A} is alkyl;

R² is aryl or heteroaryl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;

R⁴ is R⁵ or R⁶;

R⁵ is cycloalkyl, cycloalkenyl, aryl, heteroaryl or heterocyclyl;

R⁶ is alkyl which is unsubstituted or substituted with one substituent selected R⁷, R⁷O—; and

R⁷ is alkyl, aryl, heteroaryl or heterocyclyl;

wherein the moieties represented by A¹ are substituted with one or two or three substituents independently selected from the group consisting of F, Cl and O═;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —C₂ H and F;

the moieties represented by R⁷ are unsubstituted with one or two independently selected substituents independently selected from the group consisting of alkyl, F, and O═;

R⁸ is R⁹ or R¹¹;

R⁹ is cycloalkyl, heteroaryl or heterocyclyl, each of which is unsubstituted or substituted with one substituent selected from the group consisting of R¹⁰, R¹⁰SO₂—, R¹⁰C(O)—, and O═;

R¹⁰ is alkyl which is unsubstituted or substituted with one substituent selected from the group consisting of —OR¹², R¹²S—, R¹²S(O)—, R¹²SO₂—, aryl, heteroaryl and heterocyclyl; and

R¹¹ is alkyl, which is unsubstituted or substituted with heterocyclyl.

Still another embodiment pertains to compounds having formula (I), or therapeutically acceptable salts, prodrugs, salts of prodrugs, or metabolites thereof, wherein:

A¹ is phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl or 1,2,3-triazolyl;

R¹ is R², R²(CH₂)ₙNHC(O)—, R²(CH₂)ₙN(R^{1A})C(O)—or R²(CH₂)ₙOC(O)—;

n is 1, 2, 3, 4, 5 or 6;

R^{1A} is alkyl;

R² is phenyl, furanyl, imidazolyl, isothiazolyl, isoxazolyl, 1,2,3-oxadiazoyl, 1,2,5-oxadiazolyl, oxazolyl, phthalazinyl, pyrazinyl, pyrazolyl, pyridazinyl, pyridinyl, pyrimidinyl, pyrrolyl, tetrazolyl, thiazolyl, thiophenyl, triazinyl or 1,2,3-triazolyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;

R⁴ is R⁵ or R⁶;

R⁵ is cycloalkyl, cycloalkenyl, phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl or heterocyclyl;

R⁶ is alkyl which is unsubstituted or substituted with one substituent selected from the group consisting of R⁷, R⁷O—; and

R⁷ is alkyl, phenyl, furanyl, imidazolyl, isothiazolyl, isoxazolyl, 1,2,3-oxadiazoyl, 1,2,5-oxadiazolyl, oxazolyl, phthalazinyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl or heterocyclyl;

wherein the moieties represented by A¹ are substituted with one or two or three substituents independently selected from the group consisting of F, Cl, and O═;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;

the moieties represented by R⁷ are unsubstituted with one or two independently selected substituents independently selected from the group consisting of alkyl, F, and O═;

R⁸ is R⁹ or R¹¹;

R⁹ is cycloalkyl, furanyl, imidazolyl, isothiazolyl, isoxazolyl, 1,2,3-oxadiazoyl, 1,2,5-oxadiazolyl, oxazolyl, phthalazinyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl or heterocyclyl, each of which is unsubstituted or substituted with one substituent selected from the group consisting of R¹⁰, R¹⁰SO_{—, R}¹⁰C(O)—, and O═;

R¹⁰ is alkyl which is unsubstituted or substituted with one substituent selected from the group consisting of —OR¹², R¹²S—, R¹²S(O)—, R¹²SO₂—, aryl, heteroaryl and heterocyclyl; and

R¹¹ is alkyl, which is unsubstituted or substituted with heterocyclyl.

Still another embodiment pertains to compounds having formula (I) or therapeutically acceptable salts, prodrugs, salts of prodrugs, or metabolites thereof, wherein:

A¹ is phenyl;

R¹ is R², R²CH₂NHC(O)—, R²CH₂N(R^{1A})C(O)—or R²CH₂OC(O)—;

R^{1A} is C₁-C₂-alkyl;

R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;

R⁴ is R⁵ or R⁶;

R⁵ is morpholinyl, phenyl, piperazinyl, pyrazolyl, pyridinyl or thiophenyl; and

R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;

wherein the moieties represented by A¹ are substituted with one or two or three substituents independently selected from the group consisting of F, Cl, and O═;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O=, —CN, —CO₂H and F;

the moieties represented by R⁶ are unsubstituted with one or two independently selected substituents independently selected from the group consisting of C₁-alkyl and F;

R⁸ is R⁹ or R¹¹;

R⁹ is C₆-cycloalkyl, piperazinyl, piperidinyl, or thiazolyl, each of which is unsubstituted or substituted with one substituent selected from the group consisting of R¹⁰, R¹⁰SO₂—, R¹⁰C(O)—, and O═;

R¹⁰ is C₁-alkyl; and

R¹¹ is C₁-C₃-alkyl, which is unsubstituted or substituted with pyrrolidinonyl.

Still another embodiment pertains to compounds having formula (I) or therapeutically acceptable salts, prodrugs, salts of prodrugs, or metabolites thereof, wherein:

A¹ is phenyl;

R¹ is R²;

R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;

R⁴ is R⁵ or R⁶;

R⁵ is morpholinyl, phenyl, piperazinyl, pyrazolyl, pyridinyl or thiophenyl; and

R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;

wherein the moieties represented by A¹ are substituted with one or two or three substituents independently selected from the group consisting of F, Cl, and O=;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;

the moieties represented by R⁶ are unsubstituted with one or two independently selected substituents independently selected from the group consisting of C₁-alkyl and F;

R⁸ is R⁹ or R¹;

R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;

R¹⁰ is C₁-alkyl; and

R¹¹ is C₁-C₃-alkyl, which is unsubstituted or substituted with pyrrolidinonyl.

Still another embodiment pertains to compounds having formula (I) or therapeutically acceptable salts, prodrugs, salts of prodrugs, or metabolites thereof, wherein:

A¹ is phenyl;

R¹ is R²CH₂OC(O)—;

R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;

R⁴ is R⁵ or R⁶;

R⁵ is morpholinyl, phenyl, piperazinyl, pyrazolyl, pyridinyl or thiophenyl; and

R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;

wherein the moieties represented by A¹ are substituted with one or two or three substituents independently selected from the group consisting of F, Cl, and O═;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;

the moieties represented by R⁶ are unsubstituted with one or two independently selected substituents independently selected from the group consisting of C₁-alkyl and F;

R⁸ is R⁹ or R¹¹;

R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;

R¹⁰ is C₁-alkyl; and

R¹¹ is C₁-C₃-alkyl, which is unsubstituted or substituted with pyrrolidinonyl.

Still another embodiment pertains to compounds having formula (I) or therapeutically acceptable salts, prodrugs, salts of prodrugs, or metabolites thereof, wherein:

A¹ is phenyl;

R¹ is R²CH₂NHC(O)—, or R²CH₂N(R^{1A})C(O)—;

R^{1A} is C₁-C₂-alkyl;

R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;

R⁴ is R⁵ or R⁶;

R⁵ is morpholinyl, phenyl, piperazinyl, pyrazolyl, pyridinyl or thiophenyl; and

R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;

wherein the moieties represented by A¹ are substituted with one or two or three substituents independently selected from the group consisting of F, Cl, and O═;

the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;

the moieties represented by R⁶ are unsubstituted with one or two independently selected substituents independently selected from the group consisting of C₁-alkyl and F;

R⁸ is R⁹ or R¹¹;

R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;

R¹⁰ is C₁-alkyl; and

R¹¹ is C₁-C₃-alkyl, which is unsubstituted or substituted with pyrrolidinonyl.

Still another embodiment pertains to compounds of formula (I), or therapeutically acceptable salts, prodrugs, salts of prodrugs or metabolites thereof, wherein A¹ is 2-chloro-4-fluorophenyl, 2-chloro-4,5-difluorophenyl, 2,4-dichlorophenyl or 2,4,5-trifluorophenyl; and R¹ is 2,6-bis(3-(methylsulfonyl)phenyl)pyrimidin-4-yl, 6-(3-carboxyphenyl)pyrimidin-4-yl, 4-chlorophthalazin-1-yl, 6-(4-cyanophenyl)pyrimidin-4-yl, N-(3,4-dichlorobenzyl)aminocarbonyl, N,N-diethyl-6-(3-(methylsulfonyl)phenyl)-1,3,5-triazin-2-yl, 6-(3-(dimethylaminocarbonyl)phenyl)pyrimidin-4-yl or N-(3-fluorobenzyl)aminocarbonyl.

Still a further embodiment pertains to compounds of formula (I), or therapeutically acceptable salts, prodrugs, salts of prodrugs or metabolites thereof, wherein A¹ is 2-chloro-4-fluorophenyl, 2-chloro4,5-difluorophenyl, 2,4-dichlorophenyl or 2,4,5-trifluorophenyl; and R¹ is N-((3-methoxybenzyl)-N-methyl)aminocarbonyl, 4-methoxy-6-(3-(methylsulfonyl)phenyl)-1,3,5-triazin-2-yl, 6-(N-((5 -methylisoxazol-3-yl)methyl))aminopyrimidin-4-yl, 6-methyl-4-(phenylaminocarbonyl)pyrimidin-2-yl, (6-methylpyridin-2-yl)methoxycarbonyl, 6-(N-methyl-N-(pyridin-4-ylmethyl)amino)pyrimidin-4-yl, 6-(3-(methylsulfonyl)phenyl)pyrimidin-4-yl or 4-(3-(methylsulfonyl)phenyl)-1,3,5-triazin-2-yl.

Still another embodiment pertains to compounds of formula (I), or therapeutically acceptable salts, prodrugs, salts of prodrugs or metabolites thereof, wherein A¹ is 2-chloro-4-fluorophenyl, 2-chloro-4,5-difluorophenyl, 2,4-dichlorophenyl or 2,4,5-trifluorophenyl; and R¹ is 4-phenylpyrimidin-2-yl, (N-(pyridin4-ylmethyl)-N-ethyl)carbonyl, (pyridin-4-yl)methoxycarbonyl, 6-(pyridin-4-yl)pyrimidin-4-yl, 2-(thiophen-3-yl)pyrimidin4-yl, 4-(thiophen -3-yl)pyrimidin-2-yl or 6-(thiophen -3-yl)pyrimidin-4-yl.

According to a further embodiment of the present invention, there is provided a method to improve glucose tolerance in type II diabetes by administering a therapeutically effective amount of a compound of formula (I). According to other embodiments of the present invention, there is provided methods for treating type II diabetes, insulin resistance, hyperinsulinemia, impaired glucose tolerance, obesity, hypercholesterolemia, or hypertriglyceridemia by administering a therapeutically effective amount of a compound of formula (I).

According to still another embodiment, the present invention is directed to a pharmaceutical composition including a therapeutically effective amount of a compound of formula (I) in combination with a pharmaceutically suitable carrier. Another embodiment of the present invention provides a method of inhibiting DPP-IV by administering a therapeutically effective amount of a compound of formula (I). A further embodiment of the present invention provides a method of treating various disorders by inhibiting DPP-IV by administering a therapeutically effective amount of a compound of formula (I).

According to one embodiment of the present invention there is provided a method of treating diabetes by administering a therapeutically effective amount of a compound of formula (I). According to another embodiment of the present invention there is provided a method of treating type II diabetes by administering a therapeutically effective amount of a compound of formula (I). An additional embodiment of the present invention provides a method of treating hyperglycemia by administering a therapeutically effective amount of a compound of formula (I). A further embodiment of the present invention provides a method of treating Syndrome X by administering a therapeutically effective amount of a compound of formula (I). According to another embodiment of the present invention there is provided a method of treating hyperinsulinemia by administering a therapeutically effective amount of a compound of formula (I). According to still another embodiment of the present invention there is provided a method of treating obesity by administering a therapeutically effective amount of a compound of formula (I). Another embodiment of the present invention provides a pharmaceutical composition including a therapeutically effective amount of a compound of formula (I) in combination with a pharmaceutically suitable carrier.

The present invention is also directed to a method of treating disorders mediated by DPP-IV through inhibition of enzymatic activity. Disorders known to be regulated through enzymatic activity are diabetes, especially type II diabetes, as well as hyperglycemia, Syndrome X, hyperinsulinemia, obesity, atherosclerosis, various immunomodulatory diseases, and other diseases known to those of skill in the art. Therefore, according to an embodiment of the present invention there are provided compounds and associated methods of treating diabetes, especially type II diabetes, as well as hyperglycemia, Syndrome X, hyperinsulinemia, obesity, atherosclerosis, various immunomodulatory diseases, gastrointestinal diseases and disorders, cancer, cardiovascular diseases, cerebrovascular diseases, anxiety, depression, insomnia, cognitive disorders, diseases and disorders of the central nervous system, inflammation and inflammatory diseases, respiratory diseases and disorders, musculoskeletal disorders, osteoporosis, and menopausal symptoms and disorders.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, ftimarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quatemized with alkyl chlorides, bromides, and iodides such as methyl, ethyl, propyl, isopropyl, butyl, lauryl, myristyl, stearyl, and the like. The present invention contemplates pharmaceutically suitable salts formed at the nitrogen of formula (I).

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamnine, N,N-dibenzylphenethylamine, 1-ephenaamine, and N,N′-dibenzylethylenediamine, ethylenediamine, ethanolaamine, diethanolamine, piperidine, piperazine, and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (I) for example, by hydrolysis in blood.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described herein and resolved by techniques well known in the art.

Geometric isomers can exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof, oils; glycols; solutions; buffering, coloring, releasing, coating, sweetening, flavoring, and perfuiming agents; and the like. These therapeutic compositions can be administered parenterally, intracistemally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents that dissolve or disperse in the injectable media.

Inhibition of DPP-IV by the compounds of the present invention can be delayed by using a liquid suspension of crystalline or amoiphous material with poor water solubility. The rate of absorption of the compounds depends upon their rate of dissolution, which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions that are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution-retarding agent, wetting agent, absorbent, or lubricant. With capsules, tablets, and pills, the excipient can also contain buffering agents. Suppositories for rectal administration can be prepared by mixing the compounds with a suitable non-irritating excipient that is solid at ordinary temperature but fluid in the rectum.

The present compounds can be microencapsulated with one or more of the excipients discussed previously. The solid dosage forms of tablets, dragees, capsules, pills, and granules can be prepared with coatings and shells such as enteric and release controlling. In these forms, the compounds can be mixed with at least one inert diluent and can optionally comprise tableting lubricants and aids. Capsules can also optionally contain opacifying agents that delay release of the compounds in a desired part ofthe intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Disorders that can be treated or prevented in a patient by administering to the patient, a therapeutically effective amount of compound of the present invention in such an amount and for such time as is necessary to achieve the desired result. The term “therapeutically effective amount,” refers to a sufficient amount of a compound of formula (I) to effectively ameliorate disorders by inhibiting DPP-IV at a reasonable benefit/risk ratio applicable to any medical treatment. The specific therapeutically effective dose level for any particular patient will depend upon a variety of factors including the disorder being treated and the severity of the disorder, the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the compounds of the present invention necessary to inhibit the action of DPP-IV in single or divided doses can be in amounts, for example, from about 0.01to 50 mg/kg body weight. In a more preferred range, compounds of the present invention inhibit the action of DPP-IV in a single or divided dose from about 0.05 to 25 mg/kg body weight. Single dose compositions can contain such amounts or submultiple doses thereof of the compounds of the present invention to make up the daily dose. In general, treatment regimens comprise administration to a patient in need of such treatment from about 1mg to about 1000 mg of the compounds per day in single or multiple doses.

Biological Data

Isolation of Rat DPP-IV

DPP-IV was purified to homogeneity (electrophoretic) from rat kidney as described in Arch. Biochem. Biophy. 1995, 323, 148-154. Rat kidney (120 g) was homogenized in 4 volumes of water and the homogenate centrifuged for 15 minutes at 1000 g. The pH of the supernatant was adjusted to 3.9 with 1M HCl and the enzyme solubilized by autolysis for 18 hours at 37° C. The pH of the supernatant collected after centrifugation was adjusted to 7.2 with 1 M Trizma base and the enzyme was precipitated with (NH₄)₂SO₄ at 90% saturation (662 g solid ammonium sulfate per liter of solution). The solubilized precipitate was chromatographed on Sephadex G-200 (1 m×5 cm) equilibrated with a 10 mM Tris-HCl buffer pH 7.5 containing NaCl at a final concentration of 0.1 M and developed from the bottom. Fractions containing enzymatic activity were pooled, chromatographed on DE-52 (16×2.5 cm) equilibrated with 10 mM Tris-HCl, pH 7.5, and eluted with a 250-mL linear 0-0.4 M NaCl gradient prepared in 10 mM Tris-HCl. DPP-IV was then resolved from other brush border peptidases by chromatography on a phenyl Sepharose column (12×2 cm) equilibrated with 25% (NH₄)₂SO₄ at saturation (144 g ammonium sulfate per liter of 0.05 M Tris-HCl, pH 7.5). The enzyme was eluted in a homogeneous form with a 200-mL linear gradient of 25-0% (NH₄)₂SO₄, prepared in 0.05 MTris HCl buffer.

Isolation of Human DPP-IV

Caco-2 cells were obtained from American Type Culture Collection (P.O. Box 3605, Manassas, Va.), cultured and maintained at 37° C. with 5% CO₂ in low glucose DMEM media supplemented with 10% Fetal Bovine Serum and antibiotic/antimycotic. In preparation for making an extract, cells were seeded at a density to achieve confluence within 7 days. The cells were cultured for an additional 14 days to allow for maximal DPP-IV expression. On the day of harvest, cells were washed once with Dulbecco's PBS and solubilized in a 10 mM NaCI containing 50 mM Tris HCl, 0.5% Nonidet P40 and 0.3 μg/mL aprotinin at pH 8.0. The extract was clarified by centrifugation at 35,000 g for 30 minutes at 4° C. Human DPP-IV was purified from this extract supernatant, using precipitation with (NH₄)₂SO₄ at 90% saturation, as described for the rat DPP-IV. Human DPP-IV was purified from this solubilized precipitate by the same procedure as described for the solubilized precipitate of rat DPP-IV. The purified enzyme was stored frozen at −70° C. as drops collected in liquid nitrogen.

Inhibition Constant Determination for DPP-IV

DPP-IV activity was determined by measuring the rate of hydrolysis of a surrogate substrate Gly-Pro-7-amido-methylcoumarin (Gly-Pro-AMC, Catalogue #G-2761, Sigma, St. Louis, Mo.). The assay is carried out at room temperature in black 96 well polypropylene or polyethylene plates in a total volume of 100 μL per well. Appropriate dilutions of the compounds are made in DMSO and then diluted ten fold into water. 10 μL of 5 concentrations of the compound of formula (I) (inhibitor) or 10% DMSO in water are added to individual wells containing 80 μL of DPP-IV diluted in assay buffer containing 25 mM HEPES (pH 7.5), 150 mM NaCl and 0.12 mg/mL BSA. After 10 minutes at room temperature, the reaction is initiated by adding 10 μL of either 280, 700, 1750, or 3500 μM Gly-Pro-AMC in water. The DPP-IV activity results in the formation of the fluorescent product amido-methylcoumarin (AMC), which is continuously monitored by excitation at 350 nm and measurement of fluorescent emission at 460 nm every 112 seconds for 37 minutes using an appropriate plate reader. The fluorescence at 460 nm is converted to nanomoles of AMC using a standard curve and the initial rate of AMC formation is calculated. For each concentration of each of the compounds of formula (I) (inhibitor) or DMSO control, the initial rates are used to fit the rectangular hyperbola of Michaelis-Menten by non-linear regression analysis (GraphPad Software Prism 3.0). The ratio of the apparent Km/Vmax vs. inhibitor concentration is plotted and the competitive Ki is calculated by linear regression to be the negative x-intercept. The uncompetitive Ki is similarly calculated from the x-intercept of the plot of the reciprocal of the apparent Vmax versus the inhibitor concentration (Comish-Bowden , A. 1995. Fundamentals of Enzyme Kinetics. Revised edition. Portland Press, Ltd., London, U.K.).

The compounds of the present invention were found to inhibit DPP-IV induced fluorescence with inhibitory constants in a range of about 0.003 μM to about 7 μM. In a preferred range, the compounds of the present invention inhibited DPP-IV induced fluorescence with inhibitory constants in a range of about of about 0.003 μM to about 1 μM; and in a more preferred range, the compounds of the present invention inhibited DPP-FV induced fluorescence with inhibitory constants in a range of about of about 0.003 μM to about 0.5 μM.

TABLE 1
DPP-IV Inhibition of compounds of the present invention (K_{IC} nM).

| | | | |
|---|---|---|---|
| 143 | 124 | 234 | 237 |
| 664 | 183 | 192 | 224 |
| 224 | 161 | 161 | 17.8 |
| 33 | 36 | 76 | 202 |
| 34 | 36 | 35 | 57 |
| 17 | 15 | 6 | 15 |
| 7 | 3 | 3.4 | 3 |
| 3 | 84 | 3 | |

As inhibitors of DPP-IV action, the compounds of the present invention are useful in treating disorders that are mediated by DPP-IV. Disorders that are mediated by DPP-IV include diabetes, type II diabetes, hyperglycemia, Syndrome X, hyperinsulinemia and obesity. Therefore, the compounds of the present invention are useful in treating the disorder of diabetes, type II diabetes, hyperglycemia, Syndrome X, hyperinsulinemia, obesity, gastrointestinal diseases and disorders, cancer, cardiovascular diseases, cerebrovascular diseases, anxiety, depression, insomnia, cognitive disorders, diseases and disorders of the central nervous system, inflammation and inflammatory diseases, respiratory diseases and disorders, musculoskeletal disorders, osteoporosis, and menopausal symptoms and disorders.

Dipeptidyl-peptidase IV (DPP-IV, EC 3.4.14.5; CD26) is a post-proline cleaving serine protease with significant homology to other alpha-beta hydroxylases (e.g. prolyl oligopeptidase). DPP-IV is found throughout the body, both circulating in plasma and as a type II membrane protein produced by a variety of tissues, including kidney, liver and intestine. DPP-IV plays a role in the cleavage of specific substrates with accessible amino-terminal Xaa-Pro- or Xaa-Ala-dipeptide sequences, resulting in their inactivation or alteration in their biological activities. Important DPP-IV substrates include growth hormone releasing hormone, glucagon-like peptides GLP-1 and 2, gastric inhibitory polypeptide (GIP) and certain chemokines like RANTES (regulated on activation, normal T cell expressed and secreted), stromal cell-derived factor, eotaxin, and macrophage-derived chemokine (Mentlein, R. Regulatory Peptides, 1999, 85, 9-24).

The DPP-IV substrate, glucagon-like peptide GLP-1, is released from L cells in the distal small intestine and colon after oral ingestion of nutrients. The active GLP-1 (7-36) amide is an incretin that increases glucose stimulated insulin secretion (Drucker, D. J. Diabetes, 1998, 47, 159-169). Other activities attributed to GLP-1 (7-36) amide include stimulation of insulin gene expression, trophic effects on pancreatic beta cells, inhibition of glucagon secretion, promotion of satiety, inhibition of food intake, and slowing of gastric emptying (Drucker, D. J. Diabetes, 1998, 47, 159-169). These effects of GLP-1 (7-36) amide contribute to glucose homeostasis and the normalization of blood glucose levels in conditions of impaired glucose tolerance. In this regard, GLP-1 (7-36) amide has been demonstrated to reduce postprandial and fasting glycemia in patients with insulin-dependent and non-insulin-dependent diabetes mellitus (Nauck, et al., Hormone Metab. Res. 2002, 29, 411-416; Gutniak et al., J. Internal Medicine, 2001, 250, 81-87; Rauchman, et al., Diabetologia. 1997, 40, 205-11; Ahren, B., BioEssays 1998, 20, 642-51). GLP-1 based therapy has therapeutic potential for the treatment of type 2 diabetes. However, active GLP-1 (7-36) amide is rapidly converted to GLP-1 (9-36) amide by DPP-IV cleavage of the amino-terminal His-Ala- dipeptide of GLP-1 (7-36) amide (Mentlein, et al., Eur. J. Biochem. 1993, 214, 829-835). The resulting GLP-1 (9-36) amide is inactive and is an antagonist of the GLP-1 receptor (Knudson, et al., Eur. J Pharnacol. 1996, 318, 429-35). The short half-life of GLP-1 (7-36) amide in the circulation (1-1.5 minutes) makes it impractical as a therapeutic agent and has led to the development of alternative strategies to enhance the anti-diabetogenic activity of GLP-1. One strategy is to increase the circulating half-life of GLP-1, by inhibiting DPP-IV activity (Deacon, et al., Diabetes 1995, 44 1126-31). Inhibition of DPP-IV in vivo increases the level of circulating GLP-1 (7-36) amide with a concomitant increase in its insulinotropic effect (Deacon, et al., Diabetes. 1998, 47, 764-9). A DPP-IV inhibitor has been demonstrated to improve glucose tolerance in non-insulin-dependent diabetes mellitus (Ahren B, et al., Diabetes Care 2002, 25, 869-875). Therefore, the compounds of the present invention, including but not limited to those specified in the examples can be used in the treatment of conditions caused by or associated with impaired glucose tolerance including the prevention or treatment of diabetes, especially non-insulin-dependent diabetes mellitus, hyperglycemia, hyperinsulinemia and metabolic syndrome (Johannsson, et al., J. Endocrinol. Invest. 1999, 22(5 Suppl), 41-6).

Striking similarities exist between the metabolic syndrome (syndrome X) and untreated growth hormone deficiency. Abdominal/visceral obesity and insulin resistance characterize both syndromes (Reaven, GM, Physiol. Rev. 1995, 75, 473-86; Johansson, et al., Metabolism 1995, 44, 1126-29). Growth hormone favorably effects some of the perturbations associated with abdominal/visceral obesity, including reduction in abdominal/visceral obesity, improved insulin sensitivity and lipoprotein metabolism and reduction in diastolic blood pressure (BalTeto-Filho, et al., J Clin. Endocrinol. Metab. 2002, 87(5), 2018-23; Colao et al., J. Clin. Endocrinol. Metab. 2002, 87(3), 1088-93; Gotherstrom, et al., J Clin. Endocrinol. Metab. 2001, 86(10), 4657-65; Johannsson, et al., J. Endocrinol. Invest. 1999, 22(5 Suppl), 41-6; Johannsson, et al., J. Clin. Endocrinol. Metab. 1997, 82(3), 727-34).

For the treatment of diabetes or Syndrome X, compounds of the present invention can be used alone, or in combination with any existing anti-diabetic agent. Agents which can be used in combination with the compounds of the present invention include, but are not limited to insulin, an insulin analog such as mecasermin and the like, an insulin secretagogue such as nateglinide and the like, a biguanide such as metformin and the like, a sulfonylurea such as chlorpropamide, glipizide, glyburide, and the like, an insulin sensitizing agent such as a PPARγ agonist such as troglitazone, pioglitazone, rosiglitazone, and the like, an α-glucosidase inhibitor such as acarbose, voglibose, miglitol and the like, an aldose reductase inhibitor such as zopolrestat and the like, a metiglinide such as repaglinide and the like, a glycogen phosphorylase inhibitor, GLP-1 or a mimetic of GLP-1 such as exendin-4, or other such anti-diabetic agents that are known to one skilled in the art. The ability of the compounds of the present invention to treat diabetes, alone or in combination with another agent, can be demonstrated according to the methods described by Zander, M.; Mustafa, T.; Toft-Nielsen, M.-B.; Madsbad, S.; Holst, J. J. in Diabetes Care 2001, 24, 720-725; or, according to the methods described herein.

DPP-IV-mediated proteolysis has been established as a major route of growth hormone releasing hormone (GHRH) degradation and inactivation (Kubiak, et al., Drug Metab. Dispos. 1989, 17, 393-7). GHRH-derivatives that are resistant to DPP-IV cleavage are more potent in increasing serum growth hormone levels when administered i.v. due to longer stability in vivo. DPP-IV inhibition would be predicted to increase GHRH levels and thus serum growth hormone levels. Therefore, the compounds of the present invention, including but not limited to those specified in the examples can be used in the treatment of conditions associated with deficiency in growth hormone including metabolic disorders (central obesity, dyslipidemia), osteoporosis and frailty of aging.

Diabetic dyslipidemia is characterized by multiple lipoprotein defects including moderately high serum levels of cholesterol and triglycerides, small LDL particles and low levels of HDL cholesterol. The dyslipidemia associated with non-insulin-dependent diabetes mellitus is improved in conjunction with improved diabetic condition following treatment with GLP-1 (Junti-Berggren, et al., Diabetes Care 1996, 19, 1200-6). DPP-IV inhibition is predicted to increase the level of circulating GLP-1 (7-36) amide and thereby would be effective in the treatment of diabetic dyslipidemia and associated complications. Therefore, the compounds of the present invention, including but not limited to those specified in the examples can be used in the treatment of hypercholesterolemia, hypertriglyceridemia and associated cardiovascular disease.

Parenteral injection of GLP-1 (7-36) amide in healthy men, obese men or patients with non-insulin-dependent diabetes mellitus has been reported to promote satiety and to suppress food intake (Flint, et al., J. Clin. Invest. 1998, 101, 515-520; Naslund, et al., Am. J. Clin. Nutr. 1998, 68, 525-530; Gutzwiller, et al., Am. J. Physiol. 1999, 276, R1541-R1544.) DPP-IV inhibition is predicted to increase the level of circulating GLP-1 (7-36) amide and thereby increases satiety in obesity and non-insulin-dependent diabetes mellitus. Therefore, the compounds of the present invention, including but not limited to those specified in the examples can be used in the treatment of obesity.

For the treatment of obesity, compounds of the present invention can be used alone, or in combination with any existing anti-obesity agent as described by Flint, A.; Raben, A.; Astrup, A.; Holst, J. J. in J. Clin. Invest. 1998, 101, 515-520 or by Toft-Nielsen, M.-B.; Madsbad, S.; Holst, J. J. in Diabetes Care 1999, 22, 1137-1143. Agents which can be used in combination with the compounds of the present invention include, but are not limited to fatty acid uptake inhibitors such as orlistat and the like, monoamine reuptake inhibitors such as sibutramine and the like, anorectic agents such as dexfenfluramine, bromociyptine, and the like, sympathomimetics such as phentermine, phendimetrazine, mazindol, and the like, thyromimetic agents, or other such anti-obesity agents that are known to one skilled in the art.

DPP-IV is expressed on a fraction of resting T cells at low density but is strongly upregulated following T-cell activation. DPP-IV can have important functions on T cells and in the immune system. Synthetic inhibitors of the enzymatic activity of CD26 have been shown to suppress certain immune reactions in vitro and in vivo. In vitro recombinant soluble DPP-IV enhances proliferative responses of peripheral blood lymphocytes to stimulation with soluble tetanus toxoid antigen. In addition, the enhancing effect requires DPP-IV enzyme activity (Tanaka, et al., Proc. Natl. Acad. Sci. 1994, 91, 3082-86; Tanaka, et al., Proc. Natl. Acad. Sci. 1993, 90, 4583). Soluble DPP-IV up-regulates the expression of the costimulatory molecule CD86 on monocytes through its dipeptidyl peptidase IV activity suggesting that soluble DPP-IV enhances T cell immune response to recall antigen via its direct effect on antigen presenting cells (Ohnuma, et al., J. Immunol. 2001, 167(12), 6745-55). Consequently, DPP-IV inhibition would be predicted to suppress certain immune responses and thus have therapeutic benefit in the treatment of immunomodulatory diseases. Therefore, the compounds of the present invention, including but not limited to those specified in the examples can be used in the treatment of rheumatoid arthritis, multiple sclerosis, scleraderma, chronic inflammatory bowel disease or syndrome and allograft rejection in transplantation.

Chemokine receptors, especially CCR5 and CXCR4, act as cofactors for HIV-1entry into CD4+ cells and their corresponding ligands can suppress HIV entry and thus replication. The CXC chemokine, stromal cell derived factor-1 (SDF-1 ) is a chemokine for resting T-lymphocytes and monocytes. SDF-1 exists as two splice variants, SDF-1alpha and SDF-1beta that differ by four additional C-terminal residues in SDF-1beta. Truncation of the N-terminal Lys-Pro- residues from both SDF-1 alpha and SDF-1 beta results in the loss of their chemotactic and antiviral activities in vitro (Ohtsuki, et al, FEBS Lett. 1998, 431, 236-40; Shioda, et al., Proc. Natl. Acad. Sci. 1998, 95(11), 6331-6; Proost, et al., FEBS Lett. 1998,432, 73-6). DPP-IV inactivates SDF-1 alpha as a ligand for CXCR⁴ that is a T cell chemotactic receptor as well as the major co-receptor for T-tropic HIV-1 strains. DPP-IV inhibition would be predicted to increase full-length SDF-1 levels and thereby suppress HIV-1entry into CXCR⁴+ cells. Therefore, the compounds of the present invention, including but not limited to those specified in the examples can be used in the treatment of HIV infection (AIDS).

Synthetic Methods

The compounds and processes of the present invention will be better understood in connection with the following synthetic schemes, which together illustrate the methods by which the compounds of the invention can be prepared. The synthesis of compounds of formula (I,) wherein the groups L₁, L₂, R₁ and R₂ are as defined above unless otherwise noted below, are exemplified below.

Abbreviations which have been used in the descriptions of the scheme and the examples that follow are: TASF, DAST for (diethylamino)sulfur trifluoride; DMSO for dimethylsulfoxide; NMP for N-methylpyrrolidinone; DMF for N,N-dimethylformamide; DME for ethylene glycol dimethylether, DCC for 1,3-dicyclohexylcarbodiimide, HATU for O-(7-azabenzotriazol-1-yl)-N, N, N′, N′-tetramethyluronium hexafluorophosphate; HBTU for O-benzotriazole-1-yl-N, N, N′, N′-tetramethyluronium hexafluorophosphate; HOAt for 1-hydroxy-7-azabenzotriazole; HOBt for 1-hydroxybenzotriazole hydrate; TBTU for 2-(1H-benzotriazol-1-yl)-1,1,3,3-tetramethyluronium tetrafluoroborate; TFA for trifluoroacetic acid; THF for tetrahydrofuran; and PS for polymer supported.

The compounds and processes of the present invention will be better understood in connection with the following synthetic schemes, which together illustrate the methods by which the compounds of the invention can be prepared. Compounds of the present invention, can be made through the these Schemes or through similar methods conducted by one skilled in the art.

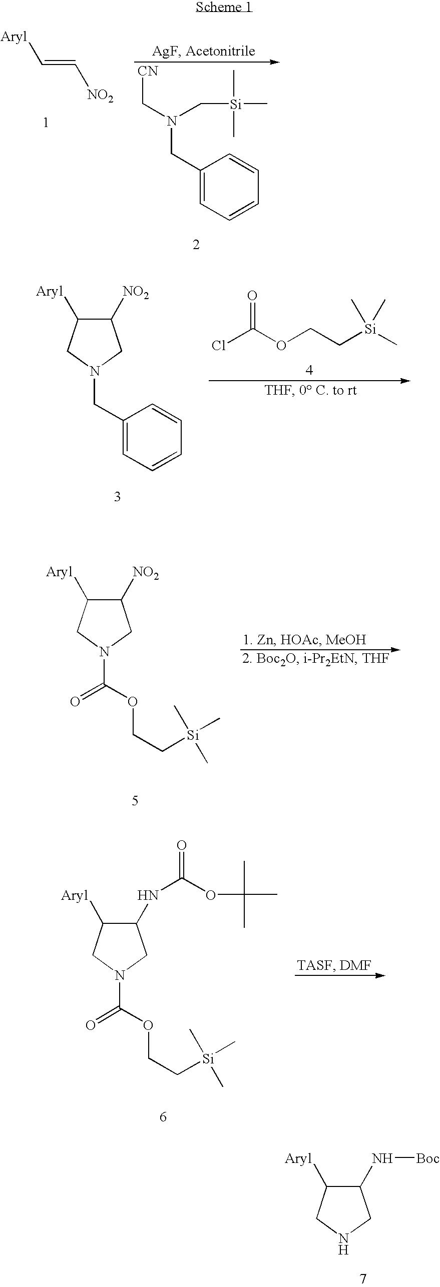

As outlined in Scheme 1, Compounds of formula 1 when treated with silver fluoride and (benzyl-trimethylsilanylmethy-amino)-acetonitrile (formula 2) will provide a compound of formula 3. Compounds of formula 3 when treated with a compound of formula 4 in a solvent such as but not limited to THF will provide a compound of formula 5. Alternatively, the conversion of compound of formula 3 to a compound of formula 5 can also be achieved in a two step sequence by removing the benzyl group using conditions know to remove N-benzyl groups or as outlined in Greenes “Protecting groups in Organic Chemistry” 3^{rd} ed. (1999, Wiley & Sons, Inc.), followed by treatment with a compound of formula 4 to provide a compound of formula 5. The reduction of the nitro group of compound of formula 5 using zinc and acetic acid in methanol followed by the protection of the formed amine as a tert-butyl carbamate using di-tertbutyl dicarbonate and diusopropylethylamine in THF will provide a compound of formula 6. The removal of the silyl protecting group using TASF (tris(dimethylamino)sulfur (trimethylsilyl)difluoride) in a solvent such as DNM will provide a compound of formula 7.

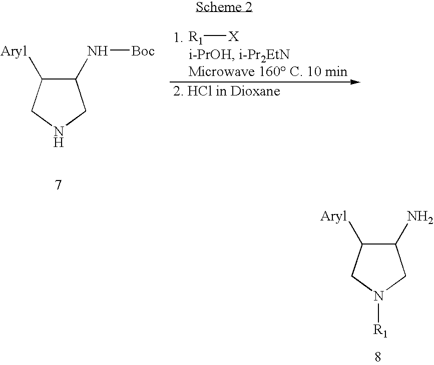

As outlined in Scheme 2, a compound of formula 7 when treated with. a compound R₁-X (wherein R₁ is described within the scope of this invention and X is a halogen such as chlorine or bromine) along with diisopropylethylamine in isopropanol under heated and microwave conditions will provide a Boc protected amine which when subjected to conditions known to remove Boc protecting groups will provide a compound of formula 8 which is representative of compounds of the present invention.

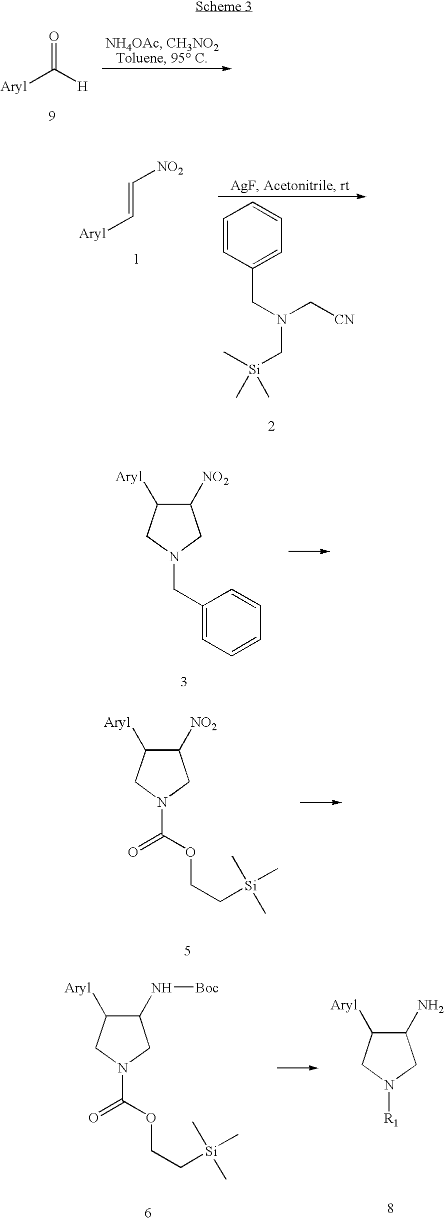

Depending on the commercial availability compounds of formula 1, which contain a variety of substituents on the aryl ring, certain compounds may need to be generated as outlined in Scheme 3. Compounds of formula 9 when treated with ammonium acetate and nitromethane in toluene under heated conditions will provide compounds of formula 1. The generated compounds of formula 1 when subjected to the same conditions outlined in Scheme 1 and Scheme 2 will provide compounds of formula 8 which are representative of compounds of the present invention.

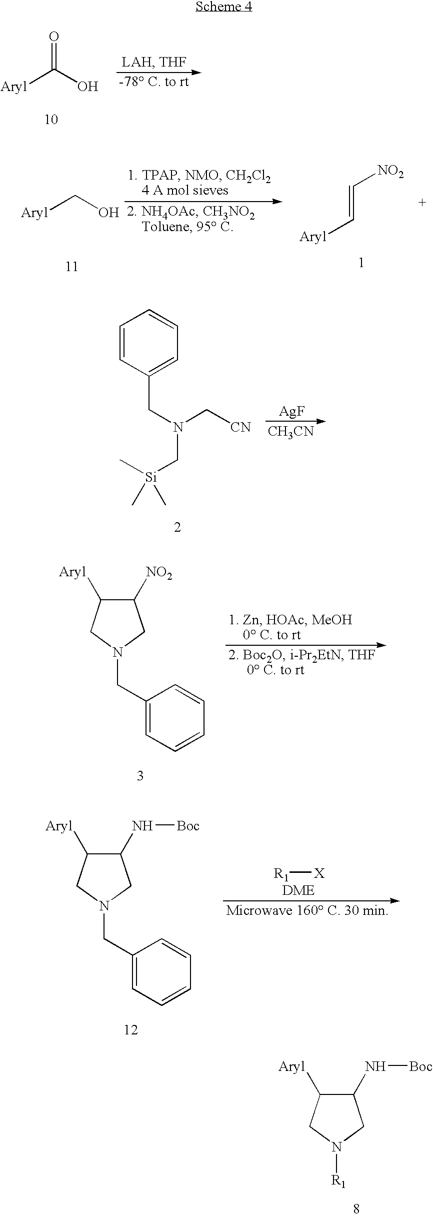

As outlined in Scheme 4, when compounds of formula 10 are treated with lithium aluminum hydride in THF from −780° C. to room temperature will provide compounds of formula 11. The alcohol functional group of compound of formula 11 when treated to oxidizing conditions such as but not limited to TPAP (tetrapropylammonium penuthenate), 4-methylmorpholine N-oxide and 4 angstrom molecular sieves in dichloromethane followed by the treatment of the formed aldehyde with ammonium acetate and nitromethane in toluene under heated conditions will provide compounds of formula 1. Due to the lack of availability of certain compounds of formula 1 when certain aryl acids of formula 10 are more readily available, they can be used to create compounds of formula 1 that contain varied functional groups. Similar to the conditions outlined above, compounds of formula 1 when treated with a compound of formula 2 and silver fluoride in acetonitrile will provide compounds of formula 3. Compounds of formula 3 when treated with zinc and acetic acid in methanol will provide an amine which when treated with di-tertbutyl dicarbonate will provide compounds of formula 12. Compounds of formula 12 when treated with a compound of formula R₁−X (wherein R₁−X is a fairly reactive electrophile) in DME under heated microwave conditions will provide compounds of formula 8 which are representative of the present invention. Although compounds of formula 12 can be directly converted to compounds of formula 8 in one step, the deprotection of the benzyl group followed by the treatment of the isolated amine with R₁−X under heated microwave conditions can be desirable based on the substituents contained within the compound and the nature of R₁−X The direct conversion of a compound of formula 12 to a compound of formula 8 by treatment with R₁−X can only work when the reagent R₁−X is fairly reactive.

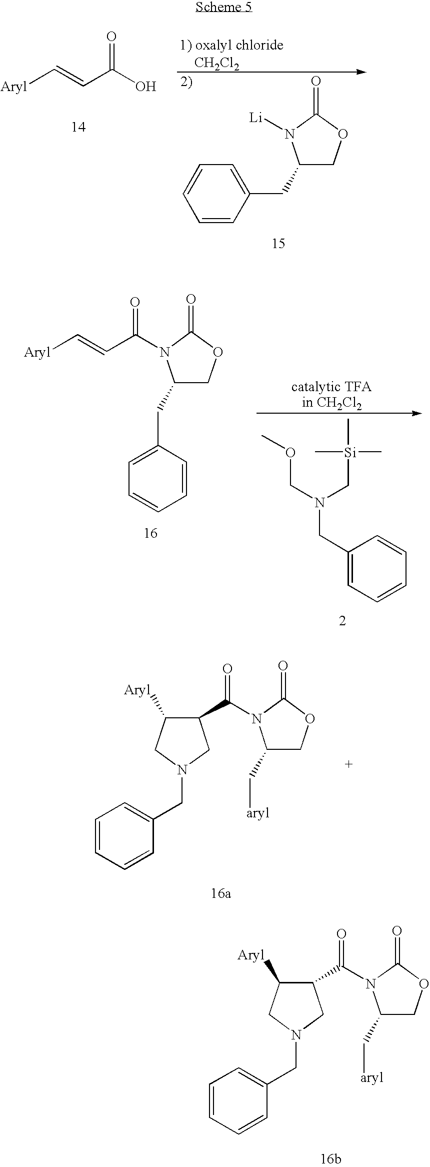

As outlined in Scheme 5, compounds of formula 14 treated with oxalyl chloride in 10 dichloromethane followed by treatment with compounds of formula 15 will provide compounds of formula 16. Compounds of formula 16 when treated with compounds of formula 2 and trifluoroacetic acid in dichloromethane will provide both compounds of formula 16a and 16b, which are readily separable by chromatographic methods known to those skilled in the art. The diastereomeric separation followed by removal of the chrial auxiliary can be utilized to create 15 enantiomerically pure compounds of the present invention.

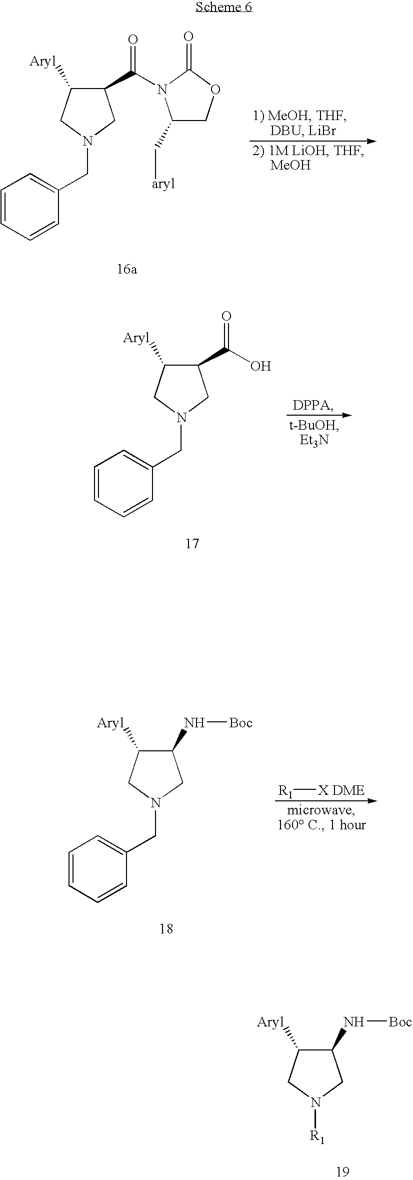

As outlined in Scheme 6, compounds of formula 16a (or 16b not shown for simplicity purposes only) when treated with DBU (1,8-diazabicyclo[5.4.0]undec-7-ene) and lithium bromide in methanol and THF followed by treatment with lithium hydroxide in THF and methanol will provide compounds of formula 17. Compounds of formula 17 when treated with diphenylphosphoryl azide, 2-methyl-2-propanol and triethylamine will provide compounds of formula 18. Compounds of formula 18 when treated with compounds of formula R₁ −X (wherein X is an appropriate leaving group such as halogen, tosyl or mesyl) in DME under heated microwave conditions will provide compounds of formula 19 which following removal of the Boc-protecting group will provide compounds which are representative of the present invention. Alternatively, the benzyl group of compounds of formula 18 can be removed using conditions known to those skilled in the art followed by treatment with R₁−X under heated microwave conditions to provide compounds of formula 19.

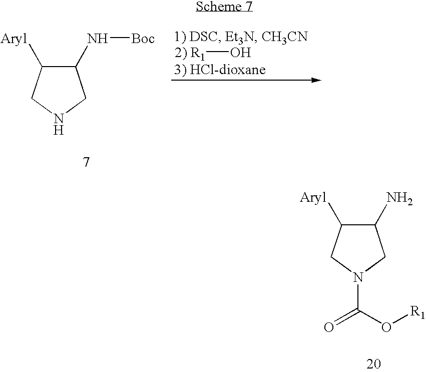

As outlined in Scheme 7, compounds of formula 7 described in the previous schemes when treated with N,N-disuccinimidyl carbonate and triethylamine in acetonitrile followed by treatment with and alcohol of formula R₁−OH followed by treatment with HCl in dioxane will provide compounds of formula 20 which are representative of compounds of the present invention.

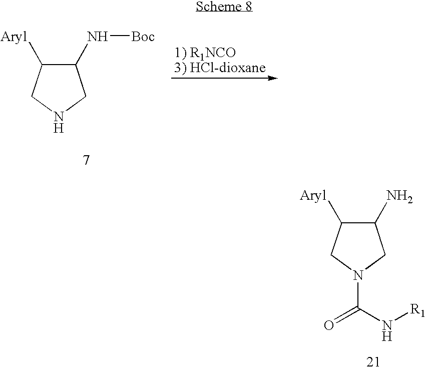

Alternatively, compounds of formula 7 when treated with an isocyanate followed by treatment with HCl in dioxane will provide compounds of formula 21 which are representative of compounds ofthe present invention.

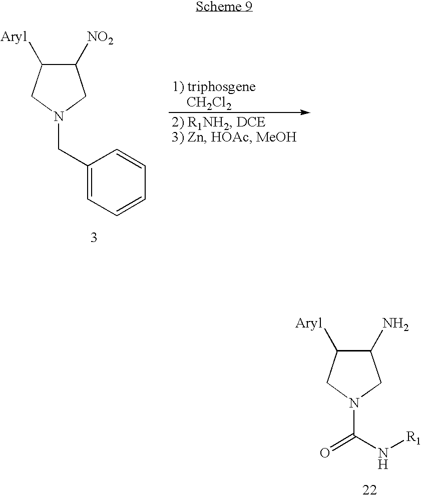

Compounds of formula 3 when treated with tiphosgene in dichlormethane followed by treatment with an amine of formula R₁NH₂ in dichloroethane followed by treatment with zinc and acetic acid in methanol will provide compounds of formula 22 which are representative of compounds of the present invention. Although this scheme represents a one pot synthetic scheme, it is contemplated that the deprotection-urea formation can be conducted separately from the reduction of the nitro group.

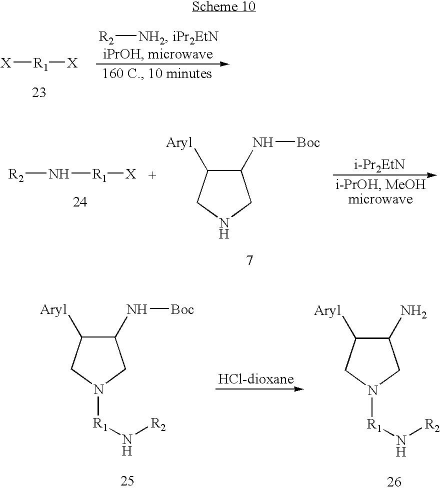

As outlined in Scheme 10, compounds of formula 23 when treated with a stoichiometrically equivalent amount of R₂−NH₂ and diisopropylethylamine in isopropanol under heated microwave conditions will provide compounds of formula 24. The treatment of compounds of formula 24 with compounds of formula 7 and diisopropylethylamine in isopropanol and methanol under heated microwave conditions will provide compounds of formula 25. Compounds of formula 25 when treated with HCl in dioxane or according to conditions that will deprotect a Boc group will provide compounds of formula 26 which are representative of compounds of the present invention.

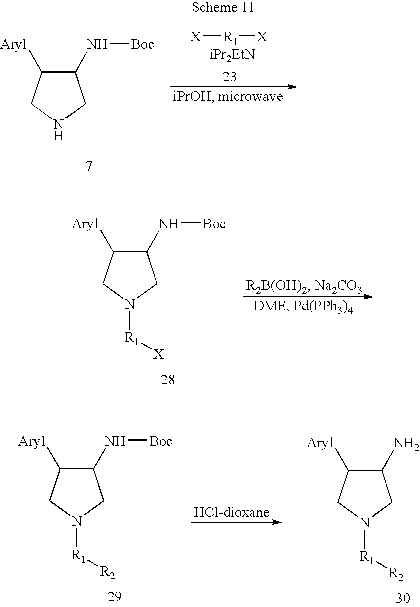

As outlined in Scheme 11, compounds of formula 7 when treated with compounds of formula 23 and diisopropylethylamine under heated microwave conditions will provide compounds of formula 28. Compounds of formula 28 when treated with a boronic acid of formula R₂B(OH)₂, sodium carbonate and tetrakis(triphenylphosphine)palladium(0) in DME will provide compounds of formula 29. Compounds of formula 29 when treated with HCl in dioxane will provide compounds of formula 30 which are representative of compounds of the present invention. Alternatively, compounds of formula 28 can be treated with a boronic acid of formula R₂B(OH)₂, dichlorobis(triphenylphosphine)palladium (II) and sodium carbonate in a mixture of DMF, MeOH, DME and water under heated microwave conditions will provide compounds of formula 29 which can be deprotected to compounds of formula 30.

The compounds and processes of the present invention will be better understood by reference to the following examples, which are intended as an illustration of and not a limitation upon the scope of the invention. Further, all citations herein are incorporated by reference.

Compounds of the invention were named by Chemdraw Ultra version 7.0.3 CambuidgeSoft Corporation, Cambridge, Mass. or were given names consistent with Chemdraw Ultra nomenclature.

The above discussion provides a factual basis for the use of the present invention descuibed herein. The present invention is further illustrated by the following non-limiting examples.

### EXPERIMENTALS

### Example 1

### 4-(2 4-dichlorophenyl)-1-(4-phenylpyrimidin-2-yl)pyrolidin-3-amine

### Example 1A

### 1-benzyl-3-(2,4-dichlorophenyl)-4-nitropoytolidine

To a solution of N-benzyl-N-(cyanomethyl)-N-[(trimethylsilyl)methyl]amine (2.1 g, 9.1 mmol) and 1,3-dichloro-5-[(E)-2-nitrovinyl]benzene (2.0 g, 9.1 mmol) in acetonitrile (20 mL), AgF (1.3 g, 10 mmol) was added. The mixture was stirred in the dark for 12 hours, diluted with methylene chloride (50 mL) and filtered through a pad of Celite. Chromatography over silica gel (5-10% ethyl acetate in hexanes) provided the title compound. ¹H NMR (300 MHz, CDCl₃) δ ppm 7.38-7.40 (m, 2H), 7.37 (s, 1H), 7.32-7.35 (m, 4H), 7.24-7.27 (m, 1H), 4.93-4.99 (m, 1H), 4.42-4.47 (m, 1H), 3.77 (d, J=12.9 Hz, 1H), 3.71 (d, J=12.9 Hz, 1H), 3.33-3.39 (m, 1H), 3.18-3.23 (m, 2H), 2.81 -2.86 (m, 1H). MS (ESI+) m/z 351 (M+H)⁺

### Example 1B

### 2-(trimethylsilyl)ethyl3-(2,4-dichlorophenyl)-4-nitropyrrolidine-1-carboxylate

A solution of 1-benzyl-3-(2,4-dichlorophenyl)-4-nitropyrTolidine (6.8 g, 19 mmol) in dry THF (20 mL) was cooled to −78° C. with a dry ice/acetone bath whereupon 2-(trimethylsilyl)ethoxycarbonyl chloride (10 g, 57 mmol) in THF (40 mL) was added. The solution was allowed to come to room temperature overnight. The mixture was filtered, concentrated under reduced pressure and purified over silica gel to provide the title compound. ¹H NMR (300 MHz, CDCl₃) δ ppm 7.47 (s, 1H), 7.28 (d, J=8.5 Hz, 1H), 7.14 (d, J=8.5 Hz, 1H), 4.93-4.97 (m, 1H), 4.42-4.45 (m, 1H), 4.26 (dd, J=7.1 Hz, J=7.1 Hz, 2H), 4.10-4.13 (m, 1H), 3.99-4.02 (m,1H), 3.70-3.85 (m, 2H), 1.06 (dd, J=7.1 Hz, J=7.1 Hz, 2H), 0.06 (s, 9H),. MS (ESI+) m/z 405 (M+H)⁺

### Example 1C

### 2-(trimethylsilyl)ethyl-3-[(tert-butoxycarbonyl)amino]-4-(2,4-dichlorophenyl)pyrrolidine-1-carboxylate

A solution of 2-(trimethylsilyl)ethyl 3-(2,4-dichlorophenyl)-4-nitropyrrolidine-1-carboxylate (5.8 g, 14 mmol) in 1:1 MeOH/AcOH was cooled to 0° C. and zinc dust (9.7 g, 150 mmol) was added in three portions. The cold bath was removed and the solution was stirred another 4 hours. The solution was concentrated under reduced pressure, taken up in ethyl acetate and washed with aqueous Na₂CO₃. The organic layer was dried with Na₂SO₄, filtered and concentrated under reduced pressure. The unpurified material was taken up in THF (35 mL) and i-Pr₂EtN (3.9 g, 30 mmol) was added. The solution was cooled to 0° C. and Boc₂O (16 mmol) was added. The solution was allowed to come to room temperature overnight with stirning. The mixture was concentrated under reduced pressure and chromatographed over silica gel to provide the title compound. ¹H NMR (300 MHz, CDCl₃) δ ppm 7.40 (s, 1H), 7.23-7.27 (m, 2H), 4.50-4.57 (m, 1H), 4.37-4.41 (m, 1H), 4.20 (t, J=8.5 Hz, 2H), 3.89-4.02 (m, 2H), 3.58-3.66 (m, 1H), 3.14-3.35 (m, 2H), 1.39 (s, 9H), 1.01 (t, J=8.5 Hz, 2H), 0.04 (s, 9H).

### Example 1D

### tert-butyl 4-(2,4-dichlorophenyl)pyrrolidin-3-ylcarbamate

To a solution of 2-(trimethylsilyl)ethyl 3-[(tert-butoxycarbonyl)amino]-4-(2,4-dichlorophenyl)pyrrolidine-1-carboxylate (3.0 g, 6.3 mmol) in DMF (12 mL), tris(dimethylamino)sulfur(trimethylsilyl)difluoride (1.9 g, 6.9 mmol) was added. The solution was stirred overnight and the solvent was removed. The residue was taken up in 0.5 M HCl and extracted with Et₂O. The organic layer was discarded and the aqueous layer was made basic with 1.0 N NaOH and extracted with ethyl acetate. The organics were dried with Na₂SO₄ and concentrated under reduced pressure. The resulting material was crystallized from ethyl acetate to provide the title compound. ¹H NMR (300 MHz, CDCl₃) δ ppm 7.38 (d, 2.0 Hz, 1H), 7.31 (d, J=8.5 Hz, 1H), 7.24 (dd, J=2.0 Hz, J=8.5 Hz, 1H), 4.72-4.74 (m, 1H), 4.23-4.26 (m, 1H), 3.46-3.57 (m, 3H), 2.85-2.98 (m, 2H), 2.43 (bs, 1H), 1.39 (s, 9). MS (ESI+) m/z 331 (M+H)⁺

### Example 1E

### 4-(2,4-dichlorophenyl)-1-(4-phenylpyrimidin-2-yl)pyrrolidin-3-amine

To a flask containing tert-butyl 4-(2,4-dichlorophenyl)pyrrolidin-3-ylcarbamate (33 mg, 0.10 mmol), 2-chloro-4-phenyl-pyrimidine (23 mg, 0.12 mmol), i-Pr₂EtN (26 mg, 0.20 mmol) and i-PrOH (0.75 mL) were added. The solution was heated by microwave (130° C., 10 minutes), concentrated under reduced pressure, and treated with 4 N HCl in dioxane (1 mL). After stirring for 1 hour, the solution was concentrated under reduced pressure and purified employing reverse phase chromatography (samples were purified by preparative HPLC on a Waters Symmetry C8 column (25 mm×100 mm, 7 μm particle size) using a gradient of 10% to 100% acetonitrile:0. 1% aqueous TFA over 8 minutes (10 minutes run time) at a flow rate of 40 mL/min) to provide the title compound. ¹H NMR (300 MHz, CD₃OD) δ ppm 8.45 (d, J=3.4 Hz, 1H), 8.30 (d, J=7.7 Hz, 2H), 7.57-7.68 (m, 6H), 7.46-7.48 (m, 1H), 4.49-4.58 (m, 2H), 7.45-7.48 (m, 2H), 3.81-3.84 (m, 1H), 4.00-4.08 (m, 1H). MS (ESI+) m/z 385 (M+H)⁺

### Example 2

### 1-(4-chlorophthalazin-1-yl)-4-(2,4-dichlorophenyl)pyrrolidin-3-amine

The title compound was prepared according to the procedure outlined in Example 1E substituting 1,4-dichlorophthalazine (24 mg, 0.12 mmol) for 2-chloro-4-phenyl-pyrimidine. ¹H NMR (300 MHz, C₅D₅N) δ ppm 8.39 (d, J=7.5 Hz, 1H), 8.06 (d, J=7.8 Hz, 1H), 7.71-7.78 (m, 3H), 7.37 (s, 1H), 7.25-7.28 (m, 1H), 5.02-5.05 (m, 1H), 4.78-4.88 (m, 3H), 4.25-4.28 (m, 1H). MS (ESI+) m/z 393 (M+H)⁺

### Example 3

### 2-[3-amino-4-(2,4-dichlorophenyl)pyrrolidin-1-yl]-6-methyl-N-phenylpyrimidine-4 -carboxamide

The title compound was prepared according to the procedure outlined in Example 1E substituting N-4-phenyl-2-chloro-6-methylpyrimidine-4-carboxylate (30 mg, 0.12 mmol) for 2-chloro4-phenyl-pyrimidine. ¹H NMR (300 MHz, C₅D₅N) δ ppm 10.44 (s, 1H), 8.72 (s, 1H), 8.21-8.25 (m, 2H), 7.60 (s, 1H), 7.51 (s, 1H), 7.40-7.43 (m, 2H), 7.30-7.32 (s, 1H), 7.15-7.18 (m, 1H), 4.54-4.56 (m, 2H), 4.46-4.51 (m, 2H), 4.35-4.39 (m, 1H), 3.74-3.78 (m, 1H), 1H), 2.37 (s, 3H). MS (ESI+) m/z 442 (M+H)⁺

### Example 4

### (6-methylpyridin-2-yl)methyl 3-amino-4-(2,4-dichlorophenyl)pyrrolidine-1-carboxylate

To a 4 mL vial, (6-methyl-pyridin-2-yl)-methanol (12 mg, 0.10 mmol) and acetonitrile (100 μL) were added. A stock solution of 1N Et₃N (300 μL) and N,N′-disuccinimidyl carbonate (26 mg, 0.10 mmol) were added, and the solution was stirred for 4 hours. Tert-butyl 4-(2,4-dichlorophenyl)pyrrolidin-3-ylcarbamate (33 mg, 0.10 mmol) was added as a solid, and the solution was stirred for another 2.5 hours. The mixture was concentrated under reduced pressure and treated with 4N HCl in dioxane (1 mL). After stirring for 1 hour, the solution was concentrated under reduced pressure and purified employing reverse phase chromatography (samples were purified by preparative HPLC on a Waters Symmetry C8 column (25 mm×100 mm, 7 um particle size) using a gradient of 10% to 100% acetonitrile:0.1% aqueous TFA over 8 minutes (10 minutes run time) at a flow rate of 40 mL/min) to provide the title compound. ¹H NMR (300 MHz, C₅D₅N) δ ppm 7.53-7.56 (m, 2H), 7.48 (bs, 2H), 7.31-7.39 (m, 2H), 7.24-7.27 (m, 1H), 7.00-7.03 (m, 1H), 5.39-5.48 (m, 2H), 4.51-4.62 (m, 2H), 4.40-4.48 (m, 2H), 4.22-4.33 (m, 2H), 3.58-3.73 (m, 2H), 2.48 (s, 1H). MS (ESI+) m/z 380 (M+H)⁺

### Example 5

### pyridin4-ylmethyl 3-amino4-(2,4-dichlorophenyl)pyrrolidine-1-carboxylate

The title compound was prepared according to the procedure outlined in Example 4 substituting pyridin-4-yl-methanol (11 mg, 0.10 mmol) for (6-methyl-pyridin-2-yl)-methanol. ¹H NMR (300 MHz, C₅D₅N) δ ppm 8.88-8.93 (m, 2H), 7.75-7.87 (m, 1H), 7.20-7.54 (m, 6H), 5.23-5.31 (m, 2H), 4.21 -4.57 (m, 4H), 3.52-3.65 (m, 2H). MS (ESI+) m/z 366 (M+H)⁺

### Example 6

### 3-amino-4-(2,4-dichlorophenyl)-N-(3-fluorobenzylpyrrolidine-1-carboxamide

To a 20 mL vial was added a solution of tert-butyl 4-(2,4-dichlorophenyl)pyrrolidin-3-ylcarbamate (33 mg, 0.10 mmol) in CH₂Cl₂ (0.25 mL). A solution of 3-fluorobenzyl isocyanate (20 mg, 0.13 mmol) in acetonitrile (0.25 mL) was added followed by i-Pr₂EtN (14 mg, 0.11 mmol) in DCM (0.25 mL). The solution was shaken at room temperature overnight. The mixture was concentrated under reduced pressure and 4N HCl in dioxane (1 mL) was added. The solution was shaken at room temperature overnight, concentrated under reduced pressure and purified employing reverse phase chromatography (samples were purified by preparative HPLC on a Waters Symmetry C8 column (25 mm×100 mm, 7um particle size) using a gradient of 10% to 100% acetonitrile: 0.1% aqueous TFA over 8 minutes (10 minutes run time) at a flow rate of 40 mL/minutes) to provide the title compound. ¹H NMR (300 MHz, CD₃OD) δ ppm 7.56 (d, J=2.2 Hz, 1H), 7.40-7.45 (m, 2H), 7.29-7.33 (m, 1H), 7.12-7.13 (m, 1H), 7.05-7.07 (m, 1H), 6.93-6.97 (m, 1H), 4.37 (s, 2H), 3.93-4.05 (m,4H), 3.51-3.54 (m, 1H), 3.46-3.49 (m, 1H).

### Example 7

### 3-amino-N-(3,4-dichlorobenzyl)4-(2,4-dichlorophenyl)pyrrolidine-1-carboxamide

The title compound was prepared according to the procedure outlined in Example 6 substituting 3,4-dichlorobenzyl isocyanate (26 mg, 0.13 mmol) for 3-fluorobenzyl isocyanate. ¹H NMR (300 MHz, CD₃OD) δ ppm 7.56 (d, J=1.9 Hz, 1H), 7.48 (d, J=2.2 Hz, 1H), 7.39-7.46 (m, 3H), 7.23-7.25 (m, 1H), 4.33 (s, 2H), 3.91-4.02 (m, 4H), 3.50-3.52 (m, 1H), 3.43-3.47 (m, 1H).

### Example 8

### 3-amino-4-(2,4-dichlorophenyl)-N-(3-methoxybenzyl)-N-methylpyrrolidine-1-carboxamide

A solution of 1-benzyl-3-(2,4-dichlorophenyl)-4-nitropyrrolidine (1.5 g, 4.3 mmol) in dry CH₂Cl₂ (5 mL) was cooled to 0° C. and triphosgene (500 mg, 1.7 mmol) in CH₂Cl₂ was added over the course of 30 minutes. The reaction was quenched with H₂O and extracted with CH₂Cl₂. The organics were then washed with saturated aqueous NaHCO₃, dried, filtered and concentrated under reduced pressure. The crude material was divided into 10 portions (0.43 mmol each), and (3-methoxy-benzyl)methyl-amine (45 mg, 0.30 mmol), Et₃N (30 mg, 0.30 mmol) and CH₂Cl₂ (1 mL) were added to a single portion. The solution was stirred for 4 hours and concentrated under reduced pressure. The unpurified material was taken up in 1:1 MeOH/AcOH (2 ml) and zinc dust (200 mg, 3 mmol) was added. After stirring 4 hours, the solution was carefully quenched with saturated aqueous NaHCO₃ and extracted with several portions of EtOAc. The organics were combined, concentrated under reduced pressure and purified employing reverse phase chromatography (samples were purified by preparative HPLC on a Waters Symmetry C8 column (25 mm×100 mm, 7 um particle size) using a gradient of 10% to 100% acetonitrile: 0.1% aqueous TFA over 8 minutes (10 minutes run time) at a flow rate of 40 mL/minutes) to provide the title compound. ¹H NMR (300 MHz, d6-DMSO) δ ppm 7.60-7.61 (m, 1H), 7.39-7.43 (m, 2H), 7.24-7.27 (m, 1H), 6.82-6.85 (m, 3H), 4.30-4.34 (m, 2H), 3.73-3.84 (m, 4H), 3.47-3.54 (m, 2H), 3.27-3.34 (m, 2H), 3.14-3.18 (m, 1H), 2.67-2.73 (s, 3H). MS (ESI+) m/z 408 (M+H)⁺

### Example 9

### 3-amino4-(2,4-dichlorophenyl)-N-(pyridin-4-ylmethyl)-N-ethylpyrrolidine-1-carboxamide

The title compound was prepared according to the procedure outlined in Example 8 substituting (pyridin-4-ylmethyl)ethyl amine (14 mg, 0.10 mmol) for (3-methoxy-benzyl)methyl amine. ¹H NMR (300 MHz, d6-DMSO) δ ppm 8.49 (d, J=5.8 Hz, 2H), 7.30-7.37 (m, 3H), 7.27 (d, J=5.8 Hz, 2H), 4.36-4.39 (m, 2H), 3.75-3.79 (m, 1H), 3.64-3.67 (m, 1H), 3.48-3.53 (m, 2H),⁺3.10-3.20 (m,4H), 1.08 (t, J=7.1 Hz, 3H). MS (ESI+) m/z 393 (M+H)+

### Example 10

### 6-[3-amino-4-(2,4-dichlorophenyl)pyrrolidin-1-yl]-N-methyl-N-(pyridin-4-ylmethyl)pyrimidin-4-amine

A solution of 4,6-dichloropyrimidine (16 mg, 0.1 mmol), i-Pr₂EtN (0.26 g, 2 mmol), (methyl-pyridin-4-yl)methyl-amine (12 mg, 0.10 mmol) and isopropanol (500 μL) were heated in the microwave at 130° C. for 20 minutes. Tert-butyl 4-(2,4-dichlorophenyl)pyrrolidin-3-ylcarbamate (33 mg, 0.10 mmol) was added as a solid, and the solution was heated in the microwave at 180° C. for 1 hour. The reaction mixture was concentrated under reduced pressure and treated with 4N HCl in dioxane (1 mL). After stirring for 1 hour, the solution was concentrated under reduced pressure and purified employing reverse phase chromatography (samples were purified by preparative HPLC on a Waters Symmetry C8 column (25 mm×100 mm, 7 um particle size) using a gradient of 10% to 100% acetonitrile:0.1% aqueous TFA over 8 minutes (10 minutes run time) at a flow rate of 40 mL/minutes) to provide the title compound. ¹H NMR (300 MHz, d6-DMSO) δ ppm 8.62 (d, J=6.2 Hz, 2H), 8.16 (s, 1H), 7.71-7.72 (m, 1H), 7.62-7.54 (m, 3H), 7.45 (d, J=5.8 Hz, 2H), 4.20-4.30 (m, 4H), 4.97 (s, 2H), 3.57-3.59 (m, 1H), 3.44-3.47 (m, 1H), 3.09 (s, 3H). MS (ESI+) m/z 429 (M+H)⁺

### Example 11

### 6-[3-amino-4 -(2,4-dichlorophenyl)pyrrolidin-1-yl]-N- [(5 -methylisoxazol-3-yl)methyl]pyrimidin-4-amine

The title compound was prepared according to the procedure outlined in Example 10 substituting (5-methyl-isoxazol-3-yl)methyl amine (11 mg, 0.10 mmol) for (methyl-pyridin-4-yl)methyl amine. ¹H NMR (300 MHz, d6-DMSO) δ ppm 8.27 (s, 1H), 8.10-8.11 (m, 1H), 7.72-7.73 (m, 1H), 7.50-7.53 (m, 2H), 6.14-6.17 (s, 1H), 5.65 (s, 1H), 4.55 (d, 6.1 Hz, 2H), 3.90-4.20 (m, 4H), 3.40-3.60 (m, 2H), 2.36-2.37 (m, 3H). MS (ESI+) m/z 419 (M+H)⁺

### Example 12

### 4-(2,4-dichlorophenyl)-1-(6-thien-3-ylpyrimidin-4-yl)pyrrolidin-3-amine

### Example 12A

### tert-butyl 1-(6-chloropyrimidin-4-yl)-4-(2,4-dichlorophenyl)pyrrolidin-3-ylcarbamate

To a solution of tert-butyl 4-(2,4-dichlorophenyl)pyrrolidin-3-ylcarbamate (0.33 g, 1.0 mmol) in isopropanol (2 mL), i-Pr₂EtN (0.26 g, 2.0 mmol) and 4,6-dichloropyrimidine (0.18 g, 1.2 mmol) were added. The vessel was sealed and heated by microwave for 10 minutes at 150° C. The solution was concentrated under reduced pressure and chromatographed over silica gel to provide the title compound. ¹H NMR (300 MHz, CDCl₃) δ ppm 8.40 (s, 1H), 7.45 (s, 1H), 7.27-7.29 (m, 2H), 6.33 (s, 1H), 4.59-4.62 (m, 1H), 4.49-4.54 (m, 1H), 4.01-4.10 (m, 1H), 3.75-3.85 (m, 1H), 3.29-3.42 (m, 2H), 1.41 (s, 9H). MS (ESI+) m/z 443 (M+H)⁺

### Example 12B

### 4-(2,4-dichlorophenyl)-1-(6-thien-3-ylpyrimidin-4-yl)prrolidin-3-amine

A vial was charged with tert-butyl 1-(6-chloropyrimidin-4-yl)-4-(2,4-dichlorophenyl)pyrrolidin-3-ylcarbamate (44 mg, 0.10 mmol), PdCl₂(PPh₃)₂ (3.5 mg, 0.5 mol %) Na₂CO₃ (42 mg, 0.4 mmol) and 3-thiophenboronic acid (15 mg, 0.12 mmol). In a separate container, a solution of 1:1:0.9:0.3 DMF/DME/MeOH/H₂O was degassed with a stream of nitrogen. The vial containing the solids was purged with N₂, charged with degassed solvent (0.7 mL), and sealed. The vial was then heated in a microwave at 150° C. for 20 minutes. The solution was concentrated under reduced pressure and partitioned between 1N NaOH and CH₂Cl₂, and the aqueous layer was extracted with CH₂Cl₂ again. The organics were concentrated under reduced pressure and treated with 4N HCl in dioxane (1 mL). After stirring for 1 hour, the solution was concentrated under reduced pressure and purified employing reverse phase chromatography (samples were purified by preparative HPLC on a Waters Symmetry C8 column (25 mm×100 mm, 7 um particle size) using a gradient of 10% to 100% acetonitrile: 0.1% aqueous TFA over 8 minutes (10 minutes run time) at a flow rate of 40 mL/minutes) to provide the title compound. ¹H NMR (300 MHz, CD₃OD) δ ppm 8.75 (s, 1H), 8.46-8.48 (m, 1H), 7.75-7.78 (m, 2H), 7.61-7.74 (m, 2H), 7.46-7.48 (m, 1H), 7.24-7.26 (m, 1H), 4.48-4.50 (m, 1H), 4.34-4.44 (m, 3H), 3.93-3.98 (m, 2H). MS (ESI+) m/z 391 (M+H)⁺

### Example 13

### 4- {6-[3-amino4-(2,4-dichlorophenyl)pyrrolidin-1-yl]pyrimidin-4-yl}benzonitrile

The title compound was prepared according to the procedure outlined in Example 12B substituting 4-cyanophenylboronic acid (18 mg, 0.12 mmol) for 3-thiophenboronic acid. ¹H NMR (300 MHz, d6-DMSO) δ ppm 8.70 (s, 1H), 8.36 (d, J=8.5 Hz, 2H), 7.99 (d, J=8.5 Hz, 2H), 7.74 (s, 1H), 7.50-7.54 (m, 2H), 7.28-7.30 (m, 1H), 4.10-4.22 (m, 4H), 3.40-3.60 (m, 2H). MS (ESI+) m/z 410 (M+H)⁺

### Example 14

### 4-(2,4-dichlorophenyl)-1-(6-pyridin-4-ylpyrimidin-4-yl)pyrrolidin-3-amine

The title compound was prepared according to the procedure outlined in Example 12B substituting pyridin-4-ylboronic acid (15 mg, 0.12 mmol) for 3-thiophenboronic acid. ¹H NMR (300 MHz, CD₃OD) δ ppm 9.12-9.16 (m, 2H), 8.43 (s, 1H), 8.65-8.70 (m, 2H), 7.58-7.65 (m, 3H), 7.45-7.48 (m, 1H), 4.50-4.55 (m, 2H), 4.33-4.40 (m, 2H), 3.96-4.06 (m, 2H). MS (ESI+) m/z 386 (M+H)⁺

### Example 15

### 4-(2,4-dichlorophenyl)-1-(4-thien-3-ylpyrimidin-2-yl)pyrrolidin-3-amine

### Example 15A

### tert-butyl 1-(4-chloropyrimidin-2-yl)-4-(2,4-dichlorophenyl)pyrrolidin-3-ylcarbamate

To a solution of tert-butyl 4-(2,4-dichlorophenyl)pyrrolidin-3-ylcarbamate (0.33 g, 1.0 mmol) in toluene (2 mL), i-Pr₂EtN (0.26 g, 2.0 mmol) and 2,4-dichloropyrimidine (0.18 g, 1.2 mmol) were added. The vessel was sealed and heated by microwave for 10 minutes at 130° C. The solution was concentrated under reduced pressure and chromatographed over silica gel to isolate tert-butyl 1-(2-chloropyrimidin-4-yl)-4-(2,4-dichlorophenyl)pyrrolidin-3-ylcarbamate and the title compound. ¹H NMR (300 MHz, CDCl₃) δ ppm 8.19 (d, J=5.1 Hz, 1H), 7.43 (d, J=2.0 Hz, 1H), 7.32 (d, 8.4Hz, 1H), 7.28 (dd, J=2.0 Hz, J=8.4 Hz, 1H), 6.58 (d, J=5.4 Hz, 1H), 4.45-4.63 (m, 2H), 4.18-4.26 (m, 2H), 3.75-3.85 (m, 1H), 3.38-3.53 (m, 2H), 1.40 (s, 9H). MS (ESI+) m/z 444 (M+H)⁺

### Example 15B

### 4-(2,4-dichlorophenyl)-1-(4-thien-3-ylpyrimidin-2-yl)pyrolidin-3-amine

The title compound was prepared according to the procedure outlined in Example 12B substituting tert-butyl 1-(4-chloropyrimidin-2-yl)-4-(2,4 -dichlorophenyl)pyrrolidin-3-ylcarbamate (44 mg, 0.10 mmol) for tert-butyl 1-(6-chloropyrimidin-4-yl)-4-(2,4-dichlorophenyl)pyrrolidin-3-ylcarbamate. ¹H NMR (300 MHz, CD₃OD) δ ppm 8.38 (d, 5.5 Hz, 1H), 8.25-8.26 (m, 1H), 7.74-7.77 (m, 1H), 7.61 (d, 2.0 Hz, 1H), 7.40-7.52 (m, 3H), 7.14 (d, 5.5 Hz, 1H), 4.23-4.33 (m, 1H), 4.14-4.19 (m, 1H), 3.80-3.88 (m, 2H). MS (ESI+) m/z 391 (M+H)⁺

### Example 16

### 4-(2,4-dichlorophenyl)-1-(2-thien-3-ylpyrimidin4-yl)pyrolidin-3-amine

### Example 16A

### tert-butyl 1-(2-chloropyrimidin-4-yl)-4-(2,4-dichlorophenyly)pyrrolidin-3-ylcarbamate

To a solution of tert-butyl 4-(2,4-dichlorophenyl)pyrrolidin-3-ylcarbamate (0.33 g, 1.0 mmol) in toluene (2 mL), i-Pr₂EtN (0.26 g, 2.0 mmol) and 2,4-dichloropyrimidine (0.18 g, 1.2 mmol) were added. The vessel was sealed and heated by microwave for 10 minutes at 130° C. The solution was concentrated under reduced pressure and chromatographed over silica gel to isolate tert-butyl 1-(4-chloropyrimidin-2-yl)-4-(2,4-dichlorophenyl)pyrrolidin-3-ylcarbamate and the title compound. ¹ H NMR (300 MHz, CDCl₃) δ ppm 8.70 (d, J=6.1 Hz, 1H), 7.44-7.46 (m, 1H), 7.28-7.30 (m, 2H), 6.23 (d, J=4.7 Hz, 1H), 4.66-4.72 (m, 1H), 4.50 -4.54 (m, 1H), 4.21-4.35 (m, 1H), 3.90-3.99 (m, 1H), 3.79-3.86 (m, 1H), 3.29-3.57 (m, 2H), 1.41 (s, 9H). MS (ESI+) m/z 444 (M+H)⁺

### Example 16B

### 4-(2,4-dichlorophenyl)-1-(2-thien-3-ylpyrimidiny-4-yl)pyrrolidin-3-amine

The title compound was prepared according to the procedure outlined in Example 12B tert-butyl 1-(2-chloropyrimidin-4-yl)-4-(2,4-dichlorophenyl)pyrrolidin-3-ylcarbamate (44 mg, 0.10 mmol) for tert-butyl 1-(6-chloropyrimidin-4-yl)-4-(2,4-dichlorophenyl)pyrrolidin-3-ylcarbamate. ¹H NMR (300 MHz, d6DMSO) δ ppm 8.35-8.45 (m, 4H), 7.66-7.81 (m, 2H), 7.48-7.60 (m, 2H), 4.14-4.25 (m, 2H), 3.97-4.12 (m, 2H), 3.85-3.90 (m, 2H). MS (ESI+) m/z 391 (M+H)⁺

### Example 17

### 4-(2-chloro-4-fluorophenyl)-1-(6-thien-3-ylpyrimidin-4-yl)pyr-olidin-3-amine

### Example 17A

### 2-chloro-4-fluoro-1-[(E)-2-nitrovinyl]benzene

To a solution 2-chloro-4-fluorobenzaldehyde (5.5 g, 35 mmol) in acetic acid (30 mL) and nitromethane (30 mL), ammonium acetate (2.7 g, 35 mmol) was added. The solution was heated at 95° C. for 6 hours and concentrated under reduced pressure. The residue was partitioned between ether and water. The organics were dried, and the residue was crystallized from hexane to provide the title compound. ¹H NMR (300 MHz, CDCl₃) δ ppm 8.35 (d, J=13.5 Hz, 1H), 7.58-7.62 (m, 2H), 7.24-7.27 (m, 1H), 7.06-7.11 (m, 1H).

### Example 17B

### 1-benzyl-3-(2-chloro-4-fluorophenyl)-4-nitropyraolidine

To a solution of N-benzyl-N-(cyanomethyl)-N-[(trimethylsilyl)methyl]amine (0.81 g, 3.5 mmol) and 2-chloro-4-fluoro-1-[(E)-2-nitrovinyl]benzene (0.70 g, 3.5 mmol) in acetonitile (10 mL), AgF (0.48 g, 3.8 mmol) was added. The mixture was stirred in the dark for 12 hours, diluted with methylene chloride (10 mL) and filtered through a pad of Celite. Chromatography over silica gel (5-10% ethyl acetate in hexanes) provided the title compound. ¹H NMR (300 MHz, CDCl₃) δ ppm 7.42 (dd, J=5.9 Hz, J=8.6 Hz, 1H), 7.32-7.34 (m, 4H), 7.26-7.30 (m, 1H), 7.12 (dd, J=2.8 Hz, J=8.6 Hz, 1H), 6.97-7.01 (m, 1H), 4.94-4.98 (m, 1H), 4.43-4.47 (m, 1H), 3.77 (d, J=12.9 Hz, 1H), 3.71 (d, J=12.9 Hz, 1H), 3.32-3.37 (m, 1H), 3.19-3.23 (m, 2H), 2.81-2.85 (m, 1H). MS (ESI+) m/z 335 (M+H)⁺

### Example 17C

### tert-butyl 1-benzyl-4-(2-chloro-4-fluorophenyl)pyrrolidin-3-ylcarbamate

A solution of 1-benzyl-3-(2-chloro-4-fluorophenyl)4-nitropyrrolidine (0.25 g, 0.75 mmol) in 1:1 MeOH/AcOH (6 mL) was cooled to 0° C. and zinc dust (0.65 g, 10 mmol) was added in three portions. The mixture was allowed to come to room temperature with stirring over the course of 4 hours. The solution was concentrated under reduced pressure, taken up in ethyl acetate and washed with aqueous Na₂CO₃. The organic layer was dried with Na₂SO₄ and concentrated under reduced pressure. The unpurified material was taken up in THF (3 mL) and i-Pr₂EtN (0.26 g, 2 mmol) was added. The mixture was cooled to O° C. and Boc₂O (0.33 g, 1.5 mmol) was added. The ice bath was removed and the solution was stirred overnight, concentrated under reduced pressure and chromatographed over silica gel to provide the title compound. ¹H NMR (300 MHz, CDCl₃) δ ppm 7.40-7.43 (m, 1H), 7.32-7.35 (m, 4H), 7.24-7.27 (m, 1H), 7.06-7.08 (m, 1H), 6.95-6.99 (m, 1H), 4.91-4.95 (m, 1H), 4.23-4.27 (m, 1H), 3.60-3.69 (m, 3H), 3.13-3.16 (m, 1H), 3.05-3.08 (m, 1H), 2.64-2.69 (m, 1H), 2.45-2.48 (m, 1H), 1.39 (s, 9H).

### Example 17D

### tert-butyl 4-(2-chloro-4-fluorophenyl)-1-(6-chloropyrimidin-4-yl)pyrrolidin-3-ylcarbamate

To a solution of tert-butyl 1-benzyl-4-(2-chloro-4-fluorophenyl)pyrrolidin-3-ylcarbamate (0.20 g, 0.5 mmol) in DME (1 mL), 4,6-dichloropyrimidine (0.15 g, 1.0 mmol) was added. The vessel was sealed and heated by microwave for 30 minutes at 160° C. The solution was concentrated under reduced pressure and chromatographed over silica gel to provide the title compound. ¹H NMR (300 MHz, CDCl₃) δ ppm 7.33-7.38 (m, 3H), 7.06-7.10 (m, 1H), 6.94-7.01 (m, 1H), 4.25-4.28 (m, 1H), 3.64-3.73 (m, 2H), 3.10-3.15 (m, 2H), 2.50-2.58 (m, 2H), 1.38 (s, 9H).

### Example 17E

### 4-(2-chloro-4-fluorophenyl)-1-(6-thien-3-ylpyrimidin-4-yl)pyrrolidin-3-amine

The title compound was prepared according to the procedure outlined in Example 12B substituting tert-butyl 4-(2-chloro-4-fluorophenyl)-1-(6-chloropyrimidin-4-yl)pyrrolidin-3-ylcarbamate (43 mg, 0.10 mmol) for tert-butyl 4-(2,4-dichlorophenyl)-1-(6-chloropyrimidin-4-yl)pyrrolidin-3-ylcarbamate. ¹H NMR (300 MHz, C₅ND₅) δ ppm 8.89 (s, 1H), 8.47 (s, 1H), 8.00 (s, 1H), 7.74-7.67 (m, 1H), 7.53-7.55 (m, 1H, 7.20-7.23 (m, 1H), 7.08 -7.11 (m, 1H), 6.94 (s, 1H), 4.70-4.80 (m, 3H), 4.55-4.57 (m, 1H), 4.26-4.30 (m, 1H), 3.55-3.65 (m, 1H). MS (ESI+) m/z 375 (M+H)⁺

### Example 18

### 4-(2-chloro-4-fluorophenyl)-1-(6-pyridin-4-ylpyrimidin4-yl)pyrrolidin-3-amine

The title compound was prepared according to the procedure outlined in Example 12B substituting tert-butyl 4-(2-chloro-4-fluorophenyl)-1-(6-chloropyrimidin-4-yl)pyrrolidin-3-ylcarbamate (43 mg, 0.10 mmol) for tert-butyl 4-(2,4-dichlorophenyl)-1-(6-chloropyrimidin-4-yl)pyrrolidin-3-ylcarbamate and pyridin-4-ylboronic acid (15 mg, 0.12 mmol) for 3-thiopheneboronic acid. ¹H NMR (300 MHz, C₅ND₅) δ ppm 8.87-8.94 (m, 3H), 8.16-8.18 (m, 2H), 7.64-7.73 (m, 1H), 7.20-7.23 (m, 1H), 7.08-7.15 (m, 2H), 4.75-4.83 (m, 3H), 8.16-8.18 (m, 1H), 4.26-4.39 (m, 1H), 3.60-3.70 (m, 1H). MS (ESI+) m/z 370 (M+H)^{+Example} 19

### 4-(2 -chloro4,5-difluorophenyl)-1-(6-thien-3-ylpybimidin-4-yl)pylrolidin-3-amine

### Example 19A

### (2-chloro-4,5-difluorophenyl)methanol

A solution of 2-chloro-4,5-difluorobenzoic acid (5.8 g, 30 mmol) in THF (100 mL) was cooled to −78° C. and 1 M lithium aluminum hydride in THF (35 mL) was added dropwise over 20 minutes. The solution was allowed to come to room temperature over 4 hours with stirring. The solution was then cooled to 0° C. and 10% aqueous ammonium chloride (35 mL) was added slowly. The organics were decanted from the aluminum salts, and the salts were washed with several portions of ethyl ether. The organics were then concentrated under reduced pressure and chromatographed over silica gel to provide the title compound. ¹H NMR (300 MHz, CDCl₃) δ ppm 7.34-7.39 (m, 1H), 7.18-7.22 (m, 1H), 4.72 (s, 2H), 1.99 (s,1H).

### Example 19B

### 1-chloro-4,5-difluoro-2-[(E)-2-nitrovinyl]benzene

To a solution of (2-chloro-4,5-difluorophenyl)methanol (1.7 g, 22 mmol) in CH₂Cl₂, TPAP (1.0 mmol), NMO (32 mmol), and 4 A molecular sieves (10.75 g) were added. The solution was stirred and filtered. The unpurified material was added to a solution of acetic acid (50 mL), nitromethane (10 mL) and ammonium acetate (2.7 g, 35 mmol). The solution was heated at 95° C. for 4 hours and concentrated under reduced pressure. The residue was partitioned between ethyl ether and water. The organics were dried, concentrated under reduced pressure and purified over silica gel to provide the title compound. ¹H NMR (300 MHz, CDCl₃) δ ppm 8.28 (d, J=13.8 Hz, 1H), 7.50 (d, J=13.8 Hz, 1H), 7.34-7.43 (m, 2).

### Example 19C

### 1-benzyl-3-(2-chloro-4,5-difluorophenyl)-4-nitropyrrolidine

To a solution of N-benzyl-N-(cyanomethyl)-N-[(trimethylsilyl)methyl]amine (0.81 g, 3.5 mmol) and 1-chloro-4,5-difluoro-2-[(E)-2-nitrovinyl]benzene (0.76 g, 3.5 mmol) in acetonitrile (10 mL), AgF (0.48 g, 3.8 mmol) was added. The mixture was stirred in the dark for 12 hours, diluted with methylene chloride (10 mL) and filtered through a pad of Celite. Chromatography over silica gel (5-10% ethyl acetate in hexanes) provided the title compound. ¹H NMR (300 MHz, CDCl₃) δ ppm 7.33-7.38 (m, 5H), 7.30-7.32 (m, 1H), 7.20-7.29 (m, 1H), 4.87-4.91 (m, 1H), 4.37-4.41 (m, 1H), 3.77 (d, J=9.5 Hz, 1H), 3.71(d, J=9.5 Hz, 1H), 3.40-3.45 (m, 1H), 3.09-3.18 (m, 2H), 2.83-2.86 (m, 1H). MS (ESI+) m/z 353 (M+H)

### Example 19D

### tert-butyl 1-benzyl-4-(2-chloro-4,5-difluorophenyl)pyrrolidin-3-ylcarbamate

A solution of 1-benzyl-3-(2-chloro-4,5-difluorophenyl)₄-nitropyrrolidine (0.26 g, 0.75 mmol) in 1:1 MeOH/AcOH (6 mL) was cooled to 0° C. and zinc dust (90.65 g, 10 mmol) was added in three portions. The mixture was allowed to come to room temperature with stirring over the course of 4 hours. The solution was concentrated under reduced pressure, taken up in ethyl acetate and washed with aqueous Na₂CO₃. The organic layer was dried with Na₂SO₄ and concentrated under reduced pressure. The unpurified material was taken up in THF (3 mL) and i-Pr₂EtN (0.26 g, 2 mmol) was added. The mixture was cooled to 0° C. and Boc₂O (0.33 g, 1.5 mmol) was added. The ice bath was removed and the solution was stirred overnight, concentrated under reduced pressure and chromatographed over silica gel to provide the title compound. ¹H NMR (300 MHz, CDCl₃) δ ppm 7.31-7.34 (m, 5H), 7.26-7.29 (m, 1H), 7.15-7.18 (m, 1H), 4.89-4.93 (m, 1H), 4.16-4.20 (m, 1H), 3.53-3.69 (m, 3H), 3.09-3.12 (m, 2H), 2.47-2.55 (m, 2H), 1.39 (s, 9H).

### Example 19E

### tert-butyl (3R,4S)-4-(2-chloro-4,5-difluorophenyl)-1-(6-chloropyrimidin-4-yl)pyrrolidin-3-ylcarbamate

To a solution of tert-butyl 1-benzyl-4-(2-chloro-4,5-difluorophenyl)pyrrolidin-3-ylcarbamate (0.21 g, 0.5 mmol) in DME (1 mL), 4,6-dichloropyrimidine (0.15 g, 1.0 mmol) was added. The vessel was sealed and heated by microwave for 30 minutes at 160° C. The solution was concentrated under reduced pressure and chromatographed over silica gel to provide the title compound. ¹H NMR (300 MHz, CDCl₃) δ ppm 7.32-7.40 (m, 3H), 7.15-7.21 (m, 1H), 4.01-4.05 (m, 1H), 3.54-3.70 (m, 2H), 3.50-3.53 (m, 2H), 3.08-3.12 (m, 2H), 1.38 (s, 9H).

### Example 19F

### 4-(2-chloro-4,5-difluorophenyl)-1-(6-thien-3-ylpyrimidin-4-yl)pyrrolidin-3-amine

The title compound was prepared according to the procedure outlined in Example 12B substituting tert-butyl 4-(2-chloro-4,5-difluorophenyl)-1-(6-chloropyrimidin-4-yl)pyrrolidin-3-ylcarbamate (44 mg, 0.10 mmol) for tert-butyl 4-(2,4-dichlorophenyl)-1-(6-chloropynrmidin-4-yl)pyrrolidin-3-ylcarbamate. ¹H NMR (300 MHz, C₅ND₅) δ ppm 8.89 (s, 1H), 8.47-50 (m, 1H), 7.99-8.02 (m, 1H), 7.79-7.84 (m, 1H), 7.52-7.54 (m, 1H), 7.34-7.38 (m, 1H), 6.95-6.96 (m, 1H), 4.40-4.61 (m, 3H), 4.56-4.59 (m, 1H), 4.27-4.33 (m, 1H), 3.60-3.66 (m, 1H). MS (ESI+)m/z 393 (M+H)⁺

### Example 20

### (3R,4S)-4-(2-chloro-4,5-difluorophenyl)-1-(6-pyridin-4-ylpyrimidin-4-yl)pyrrolidin-3-amine

The title compound was prepared according to the procedure outlined in Example 12B substituting tert-butyl 4-(2-chloro-4,5-difluorophenyl)-1-(6-chloropyrimidin-4-yl)pyrrolidin-3-ylcarbamate (44 mg, 0.10 mmol) for tert-butyl 4-(2,4-dichlorophenyl)-1-(6-chloropyrimidin-4-yl)pyrrolidin-3-ylcarbamate and pyridin-4-ylboronic acid (15 mg, 0.12 mmol) for 3-thiopheneboronic acid. ¹H NMR (300 MHz, C5ND₅) δ ppm 8.88-8.96 (m, 3H), 8.16-8.18 (m, 2H), 7.88-7.93 (m, 1H), 7.35-7.45 (m, 1H), 7.05-7.07 (m, 1H), 4.87-4.92 (m, 3H), 4.70-4.72 (m, 1H), 4.32-4.39 (m, 1H), 3,70-3.78 (m, 1H). MS (ESI+) m/z 388 (M+H)⁺

### Example 21

### 1-(6-thien-3-ylpyrimidin-4-yl)-4-(2,4,5-trifluorophenyl)pyrrolidin-3-amine

### Example 21 A

### 1,2,4 -trifluoro-5-[(E)-2-nitrovinyl]benzene

To a solution of 2,4,5-trifluorbenzaldehyde (5.0 g, 31 mmol) in acetic acid (50 mL) and nitromethane (10 mL), ammonium acetate (2.7 g, 35 mmol) was added. The solution was heated at 95° C. for 4 hours and concentrated under reduced pressure. The residue was partitioned between ether and water. The organics were dried, concentrated under reduced pressure and purified over silica gel to provide the title compound. ¹H NMR (300 MHz, CDCl₃) δ ppm 7.96 (d, J=13.8 Hz, 1H), 7.65 (d, 13.8 Hz, 1H), 7.34-7.43 (m, 1H), 7.06-7.12 (m, 1H).

### Example 21B

### 1-benzyl-3-nitro-4-(2,4,5 -trifluorophenyl)pyrrolidine

To a solution of N-benzyl-N-(cyanomethyl)-N-[(trimethylsilyl)methyl]amine (0.81 g, 3.5 mmol) and 1,2,4-trifluoro-5-[(E)-2-nitrovinyl]benzene (0.71 g, 3.5 mmol) in acetonitrile (10 mL), AgF (0.48 g, 3.8 mmol) was added. The mixture was stirred in the dark for 12 hours, diluted with methylene chloride (10 mL) and filtered through a pad of Celite. Chromatography over silica gel (5-10% ethyl acetate in hexanes) provided the title compound. ¹H NMR (300 MHz, CDCl₃) δ ppm 7.30-7.34 (m, 4H) 7.25-7.30 (m, 1H), 7.18-7.25 (m, 1H), 6.90-6.97 (m, 1H), 4.91-4.95 (m, 1H), 4.15-4.20 (m, 1H), 3.75 (d, J=12.9, 1H), 3.69 (d, J=12.9, 1H), 3.33-3.37 (m, 1H), 3.19-3.23 (m,2H), 2.66-2.70 (m, 1H).

### Example 21 C

### tert-butyl 1-benzyl-4-(2,4,5-trifluorophenyl)pyrrolidin-3-ylcarbamate

A solution of 1-benzyl-3-nitro-4-(2,4,5-trifluorophenyl)pyrrolidine (0.25 g, 0.75 mmol) in 1:1 MeOH/AcOH (6 mL) was cooled to 0° C. and zinc dust (90.65 g, 10 mmol) was added in three portions. The mixture was allowed to come to room temperature with stirring over the course of 4 hours. The solution was concentated under reduced pressure, taken up in ethyl acetate and washed with aqueous Na₂CO₃. The organic layer was dried with Na₂SO₄, filtered and concentrated under reduced pressure. The unpurified material was taken up in THF (3 mL) and i-Pr₂EtN (0.26 g, 2 mmol) was added. The mixture was cooled to 0° C. and Boc₂O (0.33 g, 1.5 mmol) was added. The ice bath was removed and the solution was stirred overnight, concentrated under reduced pressure and chromatographed over silica gel to provide the title compound. ¹H NMR (300 MHz, CDCl₃) δ ppm 7.32-7.35 (m, 4H), 7.22-7.28 (m, 1H), 7.18-7.22 (m, 1H), 6.83-6.89 (m, 1H), 4.95-5.01 (m, 1H), 4.19-4.22 (m, 1H), 3.62-3.67 (m, 2H), 3.25-3.33 (m, 1H), 3.08-3.12 (m, 1H), 3.00-3.05 (m, 1H), 2.66-2.70 (m, 1H), 2.44-2.48 (m, 1H), 1.39 (s, 9). MS (ESI+) m/z 407 (M+H)⁺

### Example 21 D

### tert-butyl 1-(6-chloropydmidin-4-yl)-4-(2,4,5-trifluorophenyl)pyrrolidin-3-ylcarbamate

To a solution of tert-butyl 1-benzyl-4-(2,4,5-trifluorophenyl)pyrrolidin-3-ylcarbamate (0.20 g, 0.50 mmol) in DME (1 mL), 4,6-dichloropyrimidine (0.15 g, 1.0 mmol) was added. The vessel was sealed and heated by microwave for 30 minutes at 160° C. The solution was concentrated under reduced pressure and chromatographed over silica gel to provide the title compound. ¹H NMR (300 MHz, CDCl₃) δ ppm 8.39 (s, 1H), 7.08-7.10 (m, 1H), 6.95-7.00 (m, 1H), 6.25 (s, 1H), 4.82-4.89 (m, 1H), 4.41-4.46 (m, 1H), 3.45-3.60 (m, 2H), 3.21-3.40 (m, 2H), 1.40 (s, 9H).

### Example 21 E

### 1-(6-thien-3-ylpydimidin-4-yl)-4-(2,4,5-trifluorophenyl)pyrrolidin-3-amine

The title compound was prepared according to the procedure outlined in Example 12B substituting tert-butyl 4-(2,4,5-trifluorophenyl)-1-(6-chloropyrimidin-4-yl)pyrrolidin-3-ylcarbamate (43 mg, 0.10 mmol) for tert-butyl 4-(2,4-dichlorophenyl)-1-(6-chloropyrimidin-4-yl)pyrrolidin-3-ylcarbamate. ¹H NMR (300 MHz, C₅ND₅) δ ppm 8.88 (s, 1H), 8.49 (s, 1H), 8.00-8.01 (m, 1H), 7.78-7.80 (m, 1H), 7.53-7.55 (m, 1H), 7.12-7.17 (m, 1H), 6.94 (s, 1H), 4.73-4.76 (m, 3H), 4.53-4.55 (m, 1H), 4.20-4.26 (m, 1H), 3.70-3.75 (m, 1H). MS (ESI+) m/z 377 (M+H)⁺

### Example 22

### 1-(6-pyrdin-4-ylpylimidin-4-yl)-4-(2,4,5-trifluorophenyl)pyrrolidin-3-amine

The title compound was prepared according to the procedure outlined in Example 12B substituting tert-butyl 4-(2,4,5-trifluorophenyl)-1-(6-chloropyrimidin-4-yl)pyrrolidin-3-ylcarbamate (43 mg, 0.10 mmol) for tert-butyl 4-(2,4-dichlorophenyl)-1-(6-chloropyrimidin-4-yl)pyrrolidin-3-ylcarbamate and pyridin-4-ylboronic acid (15 mg, 0.12 mmol) for 3-thiopheneboronic acid. ¹H NMR (300 MHz, C₅ND₅) δ ppm 8.89-8.94 (m, 3H), 8.16-8.18 (m, 1H), 7.88-7.91 (m, 1H), 7.09-7.20 (m, 3H), 4.80-4.87 (m, 3H), 4.63-4.68 (m, 1H), 4.21-4.30 (m, 1H), 3.70-3.76 (m, 1H). MS (ESI+) m/z 372 (M+H)⁺

### Example 23

### Example 23A

### (4S)-4-benzyl-3-[(2E)-3-(2,4,5-trifluorophenyl)prop-2-enoyl]-1,3-oxazolidin-2-one

A solution of 3-(2,4,5-trifluoro-phenyl)-acrylic acid (22 g, 110 mmol) in CH₂Cl₂ (200 mL) was cooled to 0° C. and 2 M oxalyl chloride in CH₂Cl₂ (80 mL) was added over 20 minutes. A small volume of DMF (0.5 mL) was added and the solution was allowed to come to room temperature and stirred for another 4 hours. The solution was concentrated under reduced pressure and used without purification in the next step. A solution of (S)-(+)-4-benzyl-2-oxazolidinone (18 g, 100 mmol) in THF (250 mL) was cooled to −78° C., and 2.5M n-BuLi (40 mL) was added dropwise to the solution over the course of 30 minutes. After the addition, the solution was stirred another 30 minutes, and the unpurified acid chloride in 100 mL THF was added in several portions. The mixture was allowed to warm to room temperature, concentrated under reduced pressure, taken up in ethyl acetate, extracted with saturated Na₂CO₃, dried and concentrated under reduced pressure. The title compound was crystallized from ethyl acetate. ¹H NMR (300 MHz, CDCl₃) δ ppm 7.97 (d, J=16.0 Hz, 1H), 7.88 (d, J=16.0 Hz, 1H), 7.46-7.55 (m, 1H), 7.23-7.38 (m, 5H), 7.97-7.04 (m, 1H), 4.76-4.84 (m, 1H), 4.20-4.30 (m, 2H), 3.37 (dd, J=3.0 Hz, J=12.7 Hz, 1H), 2.86 (dd, J=9.5 Hz, J=13.2 Hz, 1H). MS (ESI+) m/z 363 (M+H)⁺

### Example 23B

### (4S)-4-benzyl-3-{[(3R,4S)-1-benzyl-4-(2,4,5-trifluorophenyl)pyrrolidin-3-yl]carbonyl}-1,3oxazolidin-2-one

A solution of (4S)-4-benzyl-3-[(2E)-3-(2,4,5-trifluorophenyl)prop-2-enoyl]-1,3-oxazolidin-2-one (32 g, 88 mmol) and benzyl methoxymethyl-trimethylsilanyl-methylamine (25 g, 110 mmol) in toluene (300 mL) was cooled to 0° C., after which TFA (10 g, 8.8 mmol) in DCM (100 mL) was added dropwise over 30 minutes. The solution was allowed to come to room temperature and concentrated under reduced pressure. The residue was taken up in ethyl acetate, extracted with saturated Na₂CO₃, dried and concentrated under reduced pressure. The title compound was obtained after crystallization in 20% ethyl acetate/hexanes. ¹ H NMR (300 MHz, CDCl₃) δ ppm 7.26-7.37 (m, 5H), 7.22-7.25 (m, 4H), 7.17-7.20 (m, 2H), (m, 2H), 6.81-6.91 (m, 1H), 4.65-4.73 (m, 1H), 4.08-4.26 (m, 4H), 3.59-3.80 (m, 2H), 3.24-3.35 (m, 2H), 3.05-3.09 (m, 1H), 2.70-2.82 (m, 3H). MS (ESI+) m/z 495 (M+H)⁺

### Example 23C

### (3R,4S)-1-benzyl-4-(2,4,5-trifluorophenyl)pyrrolidine-3-caiboxulic acid

A solution of (4S)-4-benzyl-3-{[(3R,4S)-1-benzyl-4-(2,4,5-trifluorophenyl)pyrrolidin-3-yl]carbonyl}-1,3-oxazolidin-2-one (6.5 g, 13 mmol) and LiBr (5.7 g, 66 mmol) in methanol (100) and THF (30 mL) was cooled to −10° C., whereupon DBU (4.0 g, 26 mmol) was added dropwise. The solution was stirred for 3 hours, and H₂O and ethyl ether were added. The aqueous layer was extracted with ether and the combined organics were washed with 1 N aqueous HCl and brine. The organics were concentrated under reduced pressure with the resulting residue being taken up in THF (30 mL), methanol (30 mL) and 1N LiOH (30 mL). The solution was heated to 50° C. and stirred for 3 hours. The solution was concentrated under reduced pressure, acidified, and extracted with ethyl acetate. The organics were pooled, dried, concentrated under reduced pressure and chromatographed over silica gel to provide the title compound. ¹H NMR (300 MHz, CDCl₃) δ ppm 7.43-7.45 (m, 2H), 7.24-7.36 (m, 3H), 7.17-7.19 (m, 1H), 6.85-6.93 (m, 1H), 4.20 (d, J=12.9 Hz, 1H), 4.05-4.13 (m, 1H), 4.00 (d, J=12.9 Hz, 1H), 3.77-3.81 (m, 1H), 3.53-3.59 (m, 1H), 3.21-3.33 (m, 2H), 2.85-2.92 (m, 1H).

### Example 23D

### tert-butyl (3 R,4S)-1-benzyl-4-(2,4,5-trifluorophenyl)pyrrolidin-3-ylcarbamate

To a solution of (3R,4S)-1-benzyl-4-(2,4,5-trifluorophenyl)pyrrolidine-3-carboxylic acid (7.0 g, 21 mmol) in tert-butanol (50 mL), triethylamine (4.2 g, 42 mmol) and diphenylphosphorylazide (6.7 g, 25 mmol) were added. The solution was heated to 95° C. for 48 hours, concentrated under reduced pressure and purified over silica gel to provide the title compound. ¹H NMR (300 MHz, CDCl₃) δ ppm 7.32-7.35 (m, 4H), 7.22-7.28 (m, 1H), 7.18-7.22 (m, 1H), 6.83-6.89 (m, 1H), 4.95-5.01 (m, 1H), 4.19-4.22 (m, 1H), 3.62-3.67 (m, 2H), 3.25-3.33 (m, 1H), 3.08-3.12 (m, 1H), 3.00-3.05 (m, 1H), 2.66-2.70 (m, 1H), 2.44-2.48 (m, 1H), 1.39 (s, 9). MS (ESI+) m/z 407 (M+H)⁺

### Example 23E

### tert-butly (3R,4S)-4-(2,4,5-trifluorophenyl)-1-(6-chloropyrimidin-4-yl)pyrrolidin-3-ylcarbamate

To a solution of tert-butyl (3R,4S)-1-benzyl-4-(2,4,5-trifluorophenyl)pyrrolidin-3-ylcarbamate (0.20 g, 0.5 mmol) in DME (1 mL), 4,6-dichloropyrimidine (0.15 g, 1.0 mmol) was added. The vessel was sealed and heated by microwave for 30 minutes at 160° C. The solution was concentrated under reduced pressure and chromatographed over silica to provide the title compound. ¹H NMR (300 MHz, CDCl₃) δ ppm 8.39 (s, 1H), 7.08-7.10 (m, 1H), 6.95-7.00 (m, 1H), 6.25 (s, 1H), 4.82-4.89 (m, 1H), 4.41-4.46 (m, 1H), 3.45-3.60 (m, 2H), 3.21-3.40 (m, 1.40 (s, 9H).

### Example 23F

### (3R,4S)-1-(6-thien-3-ylpyrimidin-4-yl)-4-(24,5 -trifluorophenyl)pyrrolidin-3-amine

The title compound was prepared according to the procedure outlined in Example 12B substituting (3R,4S)-1-(6-thien-3-ylpyrimidin-4-yl)-4-(2,4,5-trifluorophenyl)pyrrolidin-3-amine (43 mg, 0.10 mmol) for tert-butyl 4-(2,4-dichlorophenyl)-1-(6-chloropyrimidin-4-yl)pyrrolidin-3-ylcarbamate. ¹H NMR (300 MHz, C₅ND₅) δ ppm 8.88 (s, 1H), 8.49 (s, 1H), 8.00-8.01 (m, 1H), 7.78-7.80 (m, 1H), 7.53-7.55 (m, 1H), 7.12-7.17 (m, 1H), 6.94 (s, 1H), 4.73-4.76 (m, 3H), 4.53-4.55 (m, 1H), 4.20-4.26 (m, 1H), 3.70-3.75 (m, 1H). MS (ESI+) m/z 377 (M+H)⁺

### Example 24

### (3,R⁴S)-1-(6-pyridin-4-ylpyrimidin-4-yl)-4-(2,4,5-trifluorophenyl)pyrrolidin-3-amine

The title compound was prepared according to the procedure outlined in Example 12B substituting (3 R,4 S)-1-(6-thien-3-ylpyrimidin-4-yl)-4-(2,4,5-trifluorophenyl)pyrrolidin-3-amine (43 mg, 0.10 mmol) for tert-butyl 4-(2,4-dichlorophenyl)-1-(6-chloropyrimidin-4-yl)pyrrolidin-3-ylcarbamate and pyridinylboronic acid (15 mg, 0.12 mmol) for 3-thiopheneboronic acid. ¹H NMR (300 MHz, C₅ND₅) δ ppm 8.89-8.94 (m, 3H), 8.16-8.18 (m, 2H), 7.88-7.91 (m, 1H), 7.09-7.20 (m, 3H), 4.80-4.87 (m, 3H), 4.63-4.68 (m, 1H), 4.21-4.30 (m, 1H), 3,70-3.76 (m, 1H). MS (ESI+) m/z 372 (M+H)⁺

### Example 25

### 3-(6-(3R,4S)-3-amino-4-(2,4,5-trifluorophenyl)pyrrolidin-1-yl)pyrimidin-4-yl)-N,N-dimethylbenzamide

The title compound was prepared according to the procedure outlined in Example 12B substituting (3R,4S)-1-(6-thien-3-ylpyrimidin4-yl)-4-(2,4,5-trifluorophenyl)pyrrolidin-3-amine (43 mg, 0.10 mmol) for tert-butyl 4-(2,4-dichlorophenyl)-1-(6-chloropyrimidin-4-yl)pyrrolidin-3-ylcarbamate and 3-N,N-dimethylcarboxyphenylboronic acid (23 mg, 0.12 mmol) for 3-thiopheneboronic acid. ¹H NMR (300 MHz, C₅ND₅) δ ppm 8.96 (s, 1H), 8.58 (s, 1H), 8.34-8.36 (m, 1H), 7.76-7.80 (m, 1H), 7.65-7.67 (m, 1H), 7.51-7.54 (m, 1H), 7.15-7.20 (m, 1H), 7.01 (s, 1H), 4.76-4.81 (m, 1H), 4.60-4.65 (m, 1H), 4.54-4.56 (m, 1H), 4.15-4.22 (m, 1H), 3.75-3.83 (m, 1H), 3.03 (s, 3H), 2.83 (s, 3H). MS (ESI+) m/z 442 (M+H)⁺

### Example 26

### 3-(6-(3R,4S)-3-amino-4-(2,3,5,trifluorophenyl)pyrrolidin-1-yl)pyrimidin-4-yl)benzoic acid

A vial was charged with (3R,4S)-1-(6-thien-3-ylpyrimidin-4-yl)-4-(2,4,5 -trifluorophenyl)pyrrolidin-3-amine (43 mg, 0.10 mmol), PdCl₂(PPh₃)₂ (3.5 mg, 0.5 mol%), Na₂CO₃ (42 mg, 0.4 mmol) and (methyl 4-carboxyphenyl acetate)boronic acid (22 mg, 0.12 mmol). In a separate container, a solution of 1:1:0.9:0.3 DMF/DME/MeOH/H₂O was degassed with a stream of nitrogen. The vial containing the solids was purged with N₂, charged with degassed solvent (0.7 mL) and sealed. The vial was then heated in a microwave at 150° C. for 20 minutes. The solution was concentrated under reduced pressure and a 1N aqueous LiOH solution (1 mL), MeOH (1 mL) and THF (2 mL) were added. The solution was stirred for 4 hours, concentrated under reduced pressure, and treated with 4N HCl in dioxane (2 mL). After stirring for 1 hour, the solution was concentrated under reduced pressure and purified employing reverse phase chromatography (samples were purified by preparative HPLC on a Waters Symmetry C8 column (25 mm×100 mm, 7 μm particle size) using a gradient of 10% to 100% acetonitrile: 0.1% aqueous TFA over 8 minutes (10 minutes run time) at a flow rate of 40 mL/minutes) to provide the title compound. ¹H NMR (300 MHz, CD₃OD) δ ppm 8.84 (s, 1H), 8.55-8.56 (m, 1H), 8.31-8.34 (m, 1H), 8.14-8.16 (m, 1H), 7.75-7.80 (m, 1H), 7.25-7.58 (m, 1H), 7.31-7.40 (m, 1H), 7.25-7.26 (m, 1H), 4.50-4.58 (m, 1H), 4.40-4.48 (m, 1H), 4.28-4.34 (m, 1H), 4.44-4.10 (m, 1H), 3.88-3.98 (m, 2H). MS (ESI+) m/z 415 (M+H)⁺

### Example 27

The title compound was prepared according to the procedure outlined in Example 12B substituting (3R,4S)-1-(6-thien-3-ylpyrimidin-4-yl)-4-(2,4,5-trifluorophenyl)pyrrolidin-3-amine (43 mg, 0.10 mmol) for tert-butyl 4-(2,4-dichlorophenyl)-1-(6-chloropyrimidin-4-yl)pyrrolidin-3-ylcarbamate and 3-methylsulfonylphenylboronic acid (24 mg, 0.12 mmol) for 3-thiopheneboronic acid. ¹H NMR (300 MHz, d6-DMSO) δ ppm 8.85 (s, 1H), 8.46-8.48 (m, 1H), 8.13-8.15 (m, 1H), 7.85-7.89 (m, 1H), 7.78-7.80 (m, 1H), 7.61-7.66 (m, 1H), 7.36 (s, 1H), 4.33-4.38 (m, 1H), 4.20-4.22 (m, 1), 4.08-4.12 (m, 2H), 3.50-3.55 (m, 1H), 3.72-3.77 (m, 1H), 3.33 (s, 3H). MS (ESI+) m/z 449 (M+H)⁺

### Example 28

### (3R,4S)-1-{4-[3-(methylsulfonyl)phenyl]-1,3,5-triazin-2-yl}-4-(2,4,5-trifluorophenyl)pyrrolidin-3-amine

### Example 28A

### tert-butyl (3R,4S)-1-(4-chloro-1,3,5-triazin-2-yl)-4-(2,4,5-trifluorophenyl)pyrrolidin-3-ylcarbamate

To 2,4-dichloro-[1,3,5]triazine (75 mg, 0.50 mmol) in DME (10 mL) at −40° C., a solution of tert-butyl (3R,4S)-4-(2,4,5-trifluorophenyl)pyrrolidin-3-ylcarbamate (16 mg, 0.50 mmol) in DME (10 mL) was added, followed by addition of i-Pr₂EtN (0.174 mL, 1.0 mmoL). The reaction mixture was stirred at −40° C. for 2 hours and concentrated in vacuo. The title compound was used for the next step without purification.

### Example 28B

### (3R,4S)-1-{4-[3-(methylsulfonyl)phenyl[-1,3,5-triazin-2-yl}-4-(2,4,5-trifluorophenyl)pyrrolidin-3-amine

To a mixture of tert-butyl (3R,4S)-1-(4-chloro-1,3,5-triazin-2-yl)-4-(2,4,5-trifluorophenyl)pyrrolidin-3-ylcarbamate (crude, 0.5 mmoL), (3-methylsulfonylphenyl)boronic acid (110 mg, 0.55 mmoL), Na₂CO₃ (80 mg, 0.75 mmoL) and PdCl₂(PPh₃)₂ (4 mg, 0.006 mmol) in a microwave vial, 3 miL of a DMF/DME/MeOH/H₂O (1/1/0.4/0.3) was added. The vial was flushed with nitrogen, sealed and heated in microwave at 120° C. for 10 minutes. The reaction mixture was cooled to room temperature and filtered. The filtrate was evaporated, and the residue was purified by silica gel flash chromatography (30% ethyl acetate/hexanes). The material was reacted with a solution of 4N HCl in 1,4-dioxane (2 mL) at 0° C. for 0.5 hour and then room temperature for 2 hours. The reaction mixture was concentrated and purified by silica gel flash chromatography (0-15% MeOH/ethyl acetate) to provide the title compound. ¹H NMR (300 MHz, CD₃OD) δ ppm 8.92-9.03 (m, 1H), 8.67-8.83 (m, 2H), 8.09-8.21 (m, 1H), 7.71-7.87 (m, 1H), 7.43-7.59 (m, 1H), 7.21-7.36 (m, 1H), 4.24-4.48 (m, 2H), 4.02-4.15 (m, 1H), 3.50-3.90 (m, 3H), 3.15-3.20 (m, 3H). MS (ESI) m/e 450 (M+H)⁺

### Example 29

### (3,R⁴)-1-{4-methoxy-6-[3-(methylsulfonyl)phenyl]-1,3,5-triazin-2-yl}-4-(2,4,5-trifluorophenyl)pyrrolidin-3-amine

### Example 29A

### tert-butyl (3R,4S)-1-(4-chloro-6-methoxy-1,3,5-triazin-2-yl)-4-(2,4,5-trifluorophenyl)pyrrolidin-3-ylcarbamate

The title compound was prepared according to the procedure outlined in Example 28A substituting 2,4-dichloro-6-methoxy-[1,3,5]triazine for 2,4-dichloro-[1,3,5]triazine. The title compound was purified by silica gel flash chromatography (0-15% ethyl acetate/hexanes).

### Example 29B

### (3R,4S)-1-{4-methoxy-6-[3-(methylsulfonyl)phenlyl[-1,3,5-triazin-2-yl}-4-(2,4,5-trifluorophenyl)pyrrolidin-3-amine

The title compound was prepared according to the procedure outlined in Example 28B. ¹H NMR (300 MHz, CD₃OD) δ ppm 8.92-8.99 (m, 1H), 8.71 -8.77 (m, 1H), 8.11-8.20 (m, 1H), 7.72-7.83 (m, 1H), 7.47-7.59 (m, 1H), 7.27-7.38 (m, 1H), 4.16-4.54 (m, 3H), 4.06-4.12 (m, 3H), 3.70-4.01 (m, 3H), 3.14-3.20 (m, 3H). MS (ESI) m/e 480 (M+H)^{+.}

### Example 30

### 4-[(3R,4S)-3-amino-4-(2,4,5-trifluorophenyl)pyrrolidin-1-yl]-N,N-diethyl-6-[3-(methylsulfonyl)phenyl]-3,5-triazin-2-amine

### Example 30A

### tert-butyl (3R,4S)-1-[4-chloro-6-(diethylamino)-1,3,5-triazin-2-yl]-4-(2,4,5-trifluorophenyl)pyrrolidin-3-ylcarbamate

The title compound was prepared according to the procedure outlined in Example 29A substituting (4,6-dichloro-[1,3,5]triazin-2-yl)-diethyl-amine for 2,4-dichloro-[1,3,5]triazine was used. The title compound was used in the next step without purification.

### Example 30B

### 4-[(3R,4S)-3-amino-4-(2,4,5-trifluorophenyl)pyrrolidin-1-yl]-N,N-diethyl-6-[3-(methylsulfonyl)phenyl]-1,3,5-triazin-2-amine

The title compound was prepared according to the procedure outlined in Example 28B. ¹H NMR (300 MHz, CD₃OD) δ ppm 8.81 -8.96 (m, 1H), 8.58-8.73 (m, 1H), 8.10-8.18 (m, 1H), 7.71-7.83 (m, 1H), 7.24-7.60 (m, 2H), 3.36-4.57 (m, 10H), 3.17 (s, 3H), 1.20-1.36 (m, 6H). MS (ESI) m/e 521 (M+H)^{+.}

### Example 31

### (3R,4S)-1-{2,6-bis[3-(methylsulfonyl)phenyl]pyrmidin-4-yl}-4-(2,4,5-trifluorophenyl)pyrrolidin-3-amine

### Example 31A

### tert-butly (3R,4S)-1-(2,6-dichloropyrimidin-4-yl)-4-(2,4,5-trifluorophenyl)pyrrolidin-3-ylcarbamate

To a solution of teilt-butyl (3R,4S)-4-(2,4,5-trifluorophenyl)pyrrolidin-3-ylcarbamate (16 mg, 0.050 mmol) in DME (0.7 mL) at room temperature, 2,4,6-trichloro-pyrimidine (10 mg, 0.050 mmol) was added. The reaction mixture was stirred for 5 minutes and concentrated. The title compound was used in the next step without purification.

### Example 31 B

### (3R,4S)-1-{2,6-bis[3-(methylsulfonyl)phenyl]pyrimidin-4-yl}-4-(2,4,5-trifluorophenyl)pyrrolidin-3-amine

The title compound was prepared according to the procedure outlined in Example 28B. ¹H NMR (300 MHz, CD₃OD) δ ppm 8.82-8.89 (m, 2H), 8.58-8.65 (m, H), 8.09-8.17 (m, 2H), 7.97 (s, 1H), 7.78-7.87 (m, 2H), 7.49-7.61(m, 1H), 7.25-7.39 (m, 1H), 3.54-3.55 (m, 8H), 3.21 (s, 6H). MS (ESI) m/z 603 (M+H)^{+.}

It is understood that the foregoing detailed description and accompanying examples are merely illustrative and are not to be taken as limitations upon the scope of the invention. Various changes and modifications including, but not limited to, those relating to the chemical structures, substituents, derivatives, intermediates, syntheses, formulations and/or methods of use of the invention, can be made without departing from the spirit of the present invention and scope thereof

What is claimed is:

1. A compound of formula (I), 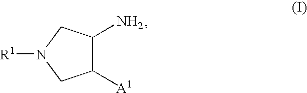
or therapeutically acceptable salts, prodrugs, salts of prodrugs, or metabolites thereof, wherein
A¹ is aryl or heteroaryl;
R¹ is R², R²C(O)—, R²OC(O)—, R²(CH₂)ₙC(O)—, R²(CH₂)ₙOC(O)—, R²NHC(O)—, R²(CH₂)ₙNHC(O)—, (R²)(R^{1A})NC(O)—, or R²(CH₂)ₙN(R^{1A})C(O)—;
n is 1, 2, 3, 4, 5 or 6;
R^{1A} is alkyl;
R² is aryl, heteroaryl or heterocyclyl, each of which is unsubstituted or substituted with one or two or three substituents independently selected from the group consisting of R⁴, R⁴O—, R⁴S—, R⁴S(O)—, R⁴SO₂—, R⁴C(O)—, R⁴OC(O)—, H₂N—, (R⁴)(H)N—, (R⁴)₂N—, NH₂C(O)—, (R⁴)(H)NC(O)—, (R⁴)₂NC(O)—, H₂NSO₂—, (R⁴)(H)NSO₂—, (R⁴)₂NSO₂—, R⁴C(O)NH—, R⁴C(O)NR⁵—, —CN, —OH, —NO₂, —CHO, —CO₂H, —CF₃, —CF₂CF₃, —OCF₃, —OCF₂CF₃, F, Cl, Br and I;
R⁴ is R⁵ or R⁶;
R⁵ is cycloalkyl, cycloalkenyl, aryl, heteroaryl or heterocyclyl;
R⁶ is alkyl which is unsubstituted or substituted with one or two or three substituents independently selected from the group consisting of R⁷, R⁷O—, R⁷S—, R⁷S(O)—, R⁷SO₂—, R⁷C(O)—, R⁷CO(O)—, R⁷OC(O)—, H₂N—, (R⁷)(H)N—, (R⁷)₂N—, NH₂C(O)—, (R⁷)(H)NC(O)—, (R⁷)₂NC(O)—, H₂NSO₂—, (R⁷)(H)NSO₂—, (R⁷ )₂NSO₂—, R⁷C(O)NH—, R⁷C(O)NR⁷ —, —CN, —OH, —CHO, —CO₂H, F, Cl, Br and I;
R⁷ is alkyl, aryl, heteroaiyl or heterocyclyl;
wherein the cycloalkyl, cycloalkenyl, aryl, heteroaryl and heterocyclyl moieties represented by A¹, R⁵ and R⁷ are independently unsubstituted or substituted with one or two or three or four or five substituents independently selected from the group consisting of R⁸, R⁸—, R⁸S—, R⁸S(O)—, R⁸SO₂—, R⁸C(O)—, R⁸OC(O)—, R⁸OC(O)O—, H₂N—, (R⁸)(H)N—, (R⁸)₂N—, NH₂C(O)—, (R⁸)(H)NC(O)—, (R⁸)₂NC(O)—, H₂NSO₂—, (R⁸)(H)NSO₂—, (R⁸)₂NSO₂—, R⁸C(O)NH—, R⁸C(O)NR⁸—, O═, —CN, —OH, —NO₂, —CHO, —CO₂H, —CF₃, —CF₂CF₃, —OCF₃, —OCF₂CF₃, F, Cl, Br and I;
R⁸ is R⁹ or R¹¹—;
R⁹ is cycloalkyl, cycloalkenyl, aryl, heteroaryl or heterocyclyl, each of which is unsubstituted or substituted with one or two or three or four substituents independently selected from the group consisting of R¹⁰, R¹⁰O—, R¹⁰S—, R¹⁰S(O)—, R¹⁰SO₂—, R¹⁰C(O)—, R¹⁰OC(O)—, H₂N—, (R¹⁰)(H)N—, (R¹⁰)₂N—, NH₂C(O)—, (R¹⁰)(H)NC(O)—, (R¹⁰)₂NC(O)—, H₂NSO₂—, (R¹⁰)(H)NSO₂—, (R¹⁰)₂NSO₂—, R¹⁰C(O)NH—, R¹⁰C(O)NR¹⁰—, O═, —CN, —OH, —NO₂, —CHO, —CO₂H, —CF₃, —CF₂CF₃, —OCF₃, —OCF₂CF₃, F, Cl, Br and I;
R¹⁰ is alkyl, which is unsubstituted or substituted with one substituent selected from the group consisting of —OR , R¹²S—, R¹²S(O)—, R¹²SO₂—, aryl, heteroaryl and heterocyclyl;
R¹¹ is alkyl which is unsubstituted or substituted with one substituent selected from the group consisting of —OR¹², R¹²S—, R¹²S(O)—, R¹²SO₂—, aryl, heteroaryl and heterocyclyl; and
R¹² is alkyl.

2. The compound according to claim 1 or therapeutically acceptable salts, prodrugs, salts of prodrugs, or metabolites thereof, wherein:
A¹ is aryl or heteroaryl;
R¹ is R², R²C(O)—, R²OC(O)—, R²(CH₂)ₙC(O)—, R¹²(CH₂)ₙOC(O)—, R²NHC(O)—, R²(CH₂)ₙNHC(O)—, (R²)(R^{1A})NC(O)—, or R²(CH₂)ₙN(R^{1A})C(O)—;
n is 1, 2, 3, 4, 5 or 6;
R^{1A} is alkyl;
R² is aryl, heteroaryl or heterocyclyl, each of which is unsubstituted or substituted with one or two or three substituents independently selected from the group consisting of R⁴, R⁴O—, R⁴S—, R⁴S(O)—, R⁴SO₂—, R⁴C(O)—, R⁴OC(O)—, H₂N—, (R⁴)(H)N—, (R⁴)₂N—, NH₂C(O)—, (R⁴)(H)NC(O)—, (R⁴)₂NC(O)—, H₂NSO₂—, (R⁴)(H)NSO₂—, (R⁴)₂NSO₂—, R⁴C(O)NH—, R⁴C(O)NR⁵—, —CN, —OH, —NO₂, —CHO, —CO₂H, —CF₃, —CF₂CF₃, —OCF₃, —OCF₂CF₃, F, Cl, Br and I;
R⁴ is R⁵ or R⁶
R⁵ is cycloalkyl, cycloalkenyl, aryl, heteroaryl or heterocyclyl;
R⁶ is alkyl which is unsubstituted or substituted with one or two or three substituents independently selected from the group consisting of R⁷, R⁷ O—, R⁷S—, R⁷S(O)—, R⁷SO₂—, R⁷C(O)—, R⁷OC(O)—, H₂N—, (R⁷)(H)N—, (R⁷)₂N—, NH₂C(O)—, (R⁷)(H)NC(O)—, (R⁷)₂NC(O)—, H₂NSO₂—, (R⁷)(H)NSO₂—, (R⁷)₂NSO₂—, R⁷C(O)NH—, R⁷C(O)NR⁷—, —CN, —OH, —CHO, —CO₂H, F, Cl, Br and I; and
R⁷ is alkyl, aryl, heteroaryl or heterocyclyl;
wherein the moieties represented by A¹ are substituted with one or two or three substituents independently selected from the group consisting of F and Cl;
the moieties represented by R⁵ are unsubstituted or substituted with one substituent selected from the group consisting of R⁸SO₂—, R⁸C(O)—, (R⁸ )₂NC(O)—, (R⁸)(H)NSO₂—, O═, —CN, —CO₂H and F;
the moieties represented by R⁷ are unsubstituted with one or two independently selected substituents independently selected from the group consisting of alkyl, F, and O═;
R⁸ is R⁹ or R¹¹;
R⁹ is cycloalkyl, heteroaryl or heterocyclyl, each of which is unsubstituted or substituted with one substituent selected from the group consisting of O═, R¹⁰, R¹⁰SO₂—, R¹⁰C(O)—and R¹⁰CO(O)—;
R¹⁰ is alkyl which is unsubstituted or substituted with one substituent selected from the group consisting of —OR¹²—, R¹²S—, R¹²S(O)—, R¹²SO₂—, aryl, heteroaryl and heterocyclyl; and
R¹¹ is alkyl, which is unsubstituted or substituted with heterocyclyl.

3. The compound according to claim1 or therapeutically acceptable salts, prodrugs, salts of prodrugs, or metabolites thereof, wherein:
A¹ is aryl or heteroaryl;
R¹ is R², R²(CH₂)ₙNHC(O)—, R²(CH₂)ₙN(R²)C(O)—or R²(CH₂)ₙOC(O)—;
n is 1, 2, 3, 4, 5 or 6;
R^{1A} is alkyl;
R² is aryl or heteroaryl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;
R⁴ is R⁵ or R⁶;
R⁵ is cycloalkyl, cycloalkenyl, aryl, heteroaryl or heterocyclyl;
R⁶ is alkyl which is unsubstituted or substituted with one substituent selected R⁷, R⁷O—; and
R⁷ is alkyl, aryl, heteroaryl or heterocyclyl;
wherein the moieties represented by A¹ are substituted with one or two or three substituents independently selected from the group consisting of F, Cl, and O═;
the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;
the moieties represented by R⁷ are unsubstituted with one or two independently selected substituents independently selected from the group consisting of alkyl, O═, and F;
R⁸ is R⁹ or R¹¹;
R⁹ is cycloalkyl, heteroaryl or heterocyclyl, each of which is unsubstituted or substituted with one substituent selected from the group consisting of R¹⁰, R¹⁰SO₂—, R¹⁰C(O)—, and O═;
R¹⁰ is alkyl which is unsubstituted or substituted with one substituent selected from the group consisting of —OR¹², R¹²S—, R¹²S(O)—, R¹²SO₂—, aryl, heteroaryl and heterocyclyl; and
R¹¹ is alkyl, which is unsubstituted or substituted with heterocyclyl.

4. The compound according to claim 1 or therapeutically acceptable salts, prodrugs, salts of prodrugs, or metabolites thereof, wherein:
A¹ is phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl or 1,2,3-triazolyl;
R¹ is R², R²(CH₂)ₙNHC(O)—, R²(CH₂)ₙN(R^{1A})C(O)—or R²(CH₂)ₙOC(O)—;
n is 1, 2, 3, 4, 5 or 6;
R^{1A} is alkyl;
R² is phenyl, fuiranyl, imidazolyl, isothiazolyl, isoxazolyl, 1,2,3-oxadiazoyl, 1,2,5-oxadiazolyl, oxazolyl, phthalazinyl, pyrazinyl, pyrazolyl, pyridazinyl, pyridinyl, pyrimidinyl, pyrrolyl, tetrazolyl, thiazolyl, thiophenyl, triazinyl or 1,2,3-triazolyl, each of which is unsubstituted or substituted with one or two substituents independently selected from the group consisting of R⁴, R⁴O—, (R⁴)(H)N—, (R⁴)ₙN—, (R⁴)(H)NC(O)—, F, Cl, Br and I;
R⁴ is R⁵ or R⁶;
R⁵ is cycloalkyl, cycloalkenyl, phenyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl or heterocyclyl;
R⁶ is alkyl which is unsubstituted or substituted with one substituent selected from the group consisting of R⁷, R⁷O—; and
R⁷ is alkyl, phenyl, furanyl, imidazolyl, isothiazolyl, isoxazolyl, 1,2,3-oxadiazoyl, 1,2,5-oxadiazolyl, oxazolyl, phthalazinyl, pyrazinyl, pyrazolyl, pyridazinyl, pyridinyl, pyrimidinyl, pyrrolyl, tetrazolyl, thiazolyl, thiophenyl, triazinyl,1,2,3-triazolyl or heterocyclyl;
wherein the moieties represented by A¹ are substituted with one or two or three substituents independently selected fiom the group consisting of F, Cl, and O═;
the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;
the moieties represented by R⁷ are unsubstituted with one or two independently selected substituents independently selected from the group consisting of alkyl, O═, and F;
R⁸ is R⁹ or R¹¹;
R⁹ is cycloalkyl, furanyl, imidazolyl, isothiazolyl, isoxazolyl, 1,2,3-oxadiazoyl, 1,2,5-oxadiazolyl, oxazolyl, phthalazinyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl or heterocyclyl, each of which is unsubstituted or substituted with one substituent selected from the group consisting of R¹⁰, R¹⁰SO₂—, R¹⁰C(O)—, and O═;
R¹⁰ is alkyl which is unsubstituted or substituted with one substituent selected from the group consisting of —OR¹², R¹²S—, R¹²S(O)—, R¹²SO₂—, aryl, heteroaryl and heterocyclyl; and
R¹¹ is alkyl, which is unsubstituted or substituted with heterocyclyl.

5. The compound according to claim 1 or therapeutically acceptable salts, prodrugs, salts of prodrugs, or metabolites thereof, wherein:
A¹ is phenyl;
R¹ is R², R²CH₂NHC(O)—, R²CH₂N(R^{1A})C(O)—or R²CH₂OC(O)—;
R^{1A} is C₁-C₂-alkyl;
R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;
R⁴ is R⁵ or R⁶;
R⁵ is morpholinyl, phenyl, piperazinyl, pyrazolyl, pyridinyl or thiophenyl; and
R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;
wherein the moieties represented by A¹ are substituted with one or two or three substituents independently selected from the group consisting of F, Cl, and O═;
the moieties represented by R⁵ are unsubstituted or substituted with one substituent selected from the group consisting of R⁸SO—, R⁸C(O)—, (R⁸)₂NC(O)—, (R⁸)(H)NSO₂—, O═, —CN, —CO₂H and F;
the moieties represented by R⁶ are unsubstituted with one or two independently selected substituents independently selected from the group consisting of C₁-alkyl and F;
R⁸ is R⁹ or R¹¹;
R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;
R¹⁰ is C₁-alkyl; and
R¹¹ is C₁-C₃-alkyl, which is unsubstituted or substituted with pyrrolidinonyl.

6. The compound according to claim 1 or therapeutically acceptable salts, prodrugs, salts of prodrugs, or metabolites thereof, wherein:
A¹ is phenyl;
R¹ is R²;
R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;
R⁴ is R⁵ or R⁶;
R⁵ is morpholinyl, phenyl, piperazinyl, pyrazolyl, pyridinyl or thiophenyl; and
R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;
wherein the moieties represented by A¹ are substituted with one or two or three substituents independently selected from the group consisting of F, Cl, and O═;
the moieties represented by R⁵ are unsubstituted or substituted with one substituent selected from the group consisting of R⁸SO₂—, R⁸C(O)—, (R⁸)₂NC(O)—, (R⁸)(H)NSO₂—, O═, —CN, —CO₂H and F;
the moieties represented by R⁶ are unsubstituted with one or two independently selected substituents independently selected from the group consisting of C₁-alkyl and F;
R⁸ is R⁹ or R¹¹;
R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;
R¹⁰ is C₁-alkyl; and
R ¹¹ is C₁-C₃-alkyl, which is unsubstituted or substituted with pyrrolidinonyl.

7. The compound according to claim 1 or therapeutically acceptable salts, prodrugs, salts of prodrugs, or metabolites thereof, wherein:
A¹ is phenyl;
R¹ is R²CH₂OC(O)—;
R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;
R⁴ is R⁵ or R⁶;
R⁵ is morpholinyl, phenyl, piperazinyl, pyrazolyl, pyridinyl or thiophenyl; and
R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;
wherein the moieties represented by A¹ are substituted with one or two or three substituents independently selected from the group consisting of F, Cl, and O═;
the moieties represented by R⁵ are unsubstituted or substituted with one substituent selected from the group consisting of R⁸SO—, R⁸C(O)—, (R⁸)₂NC(O)—, (R⁸)(H)NSO₂—, O═, —CN, —CO₂H and F;
the moieties represented by R⁶ are unsubstituted with one or two independently selected substituents independently selected from the group consisting of C₁-alkyl and F;
R⁸ is R⁹ or R¹¹;
R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;
R¹⁰ is C₁-alkyl; and
R¹¹ is C₁-C₃-alkyl, which is unsubstituted or substituted with pyrrolidinonyl.

8. The compound according to claim 1 or therapeutically acceptable salts, prodrugs, salts of prodrugs, or metabolites thereof, wherein:
A¹ is phenyl;
R¹ is R²CH₂NHC(O)—, or R²CH₂N(R^{1A})C(O)—;
R^{1A} is C₁-C₂-alkyl;
R² is phenyl, phthalazinyl, pyridinyl, pyrimidinyl, or triazinyl, each of which is unsubstituted or substituted with one or two substituents independently selected from the group consisting of R⁴, R⁴O—, (R⁴)(H)N—, (R⁴)₂N—, (R⁴)(H)NC(O)—, F, Cl, Br and I;
R⁴ is R⁵ or R⁶;
R⁵ is morpholinyl, phenyl, piperazinyl, pyrazolyl, pyridinyl or thiophenyl; and
R⁶ is C₁-C₂-alkyl which is unsubstituted or substituted with one substituent selected from the group consisting of 1,3-benzodioxolyl, isoxazolyl, morpholinyl, phenyl, pyridinyl and (C₁-alkyl)O—;
wherein the moieties represented by A¹ are substituted with one or two or three substituents independently selected from the group consisting of F, Cl, and O═, the moieties represented by R⁵ are unsubstituted or substituted with one substituent selected from the group consisting of R⁸SO—, R⁸C(O)—, (R⁸)₂NC(O)—, (R⁸)(H)NSO₂—, O═, —CN, —CO₂H and F;
the moieties represented by R⁶ are unsubstituted with one or two independently selected substituents independently selected from the group consisting of C₁-alkyl and F;
R⁸ is R⁹ or R¹¹;
R⁹ is C₆-cycloalkyl, piperazinyl, piperidinyl, or thiazolyl, each of which is unsubstituted or substituted with one substituent selected from the group consisting of R¹⁰, R¹⁰SO₂—, and R¹⁰C(O)—;
R¹⁰ is C₁-alkyl; and
R¹¹ is C₁-C₃-alkyl, which is unsubstituted or substituted with pyrrolidinonyl.

9. The compound according to claim 1 or therapeutically acceptable salts, prodrugs, salts of prodrugs, or metabolites thereof, wherein A¹ is 2-chloro-4-fluorophenyl, 2-chloro-4,5-difluorophenyl, 2,4-dichlorophenyl or 2,4,5-trifluorophenyl; and R¹ is 2,6-bis(3-(methylsulfonyl)phenyl)pyrimidin-4-yl, 6-(3-carboxyphenyl)pyrimidin-4-yl, 4-chlorophthalazin-1-yl, 6-(4-cyanophenyl)pyrimidin-4-yl, N-(3,4-dichlorobenzyl)aminocarbonyl, N,N-diethyl-6-(3-(methylsulfonyl)phenyl)-1,3,5 -triazin-2 -yl, 6-(3-(dimethylaminocarbonyl)phenyl)pyrimidin-4-yl or N-(3-fluorobenzyl)aminocarbonyl.

10. The compound according to claim 1 or therapeutically acceptable salts, prodrugs, salts of prodrugs or metabolites thereof, wherein A¹ is 2-chloro-4-fluorophenyl, 2-chloro-4,5-difluorophenyl, 2,4-dichlorophenyl or 2,4,5-trifluorophenyl; and R¹ is N-((3-methoxybenzyl)-N-methyl)amninocarbonyl, 4-methoxy-6-(3-(methylsulfonyl)phenyl)-1,3,5-triazin-2-yl, 6-(N-((5methylisoxazol-3-yl)methyl))aminopyrimifin-4-yl, 6-methyl-4-(phenylaminocarbonyl)pyrimidin-2-yl, (6-methylpyridin-2-yl)methoxycarbonyl, 6-(N-methyl-N-(pyridin-4-ylmethyl)amino)pyrimidin-4-yl, 6-(3-(methylsulfonyl)phenyl)pyrimidin-4-yI or 4-(3-(methylsulfonyl)phenyl)-1,3,5-triazin-2 -yl.

11. The compound according to claim 1 or therapeutically acceptable salts, prodrugs, salts of prodrugs, or metabolites thereof, wherein A¹ is 2-chloro-4-fluorophenyl, 2-chloro-4,5-difluorophenyl, 2,4-dichlorophenyl or 2,4,5-trifluorophenyl; and R¹ is 4-phenylpyrimidin-2-yl,(N-(pyridin-4-ylmethyl)-N-ethyl)carbonyl, (pyridin-4-yl)methoxycarbonyl, 6-(pyridin-4-yl)pyrimidin-4-yl, 2-(thiophen -3-yl)pyrimidin-4-yl, 4-(thiophen -3-yl)pyrimidin-2-yl or 6-(thiophen -3-yl)pyrimidin-4-yl.

12. A method of inhibiting DPP-IV comprising the step of administering a therapeutically effective amount of a compound according to claim 1.

13. A method oftreating disorders by inhibiting DPP-IV comprising the step of administering a therapeutically effective amount of a compound according to claim 1.

14. A method of treating diabetes comprising the step of administering a therapeutically effective amount of a compound according to claim 1.

15. A method of treating type II diabetes comprising the step of administering a therapeutically effective amount of a compound according to claim 1.

16. A method of treating hyperglycemia comprising the step of administering a therapeutically effective amount of a compound according to claim 1.

17. A method of treating Syndrome X comprising the step of administering a therapeutically effective amount of a compound according to claim 1.

18. A method of treating hyperinsulinemia comprising the step of administering a therapeutically effective amount of a compound according to claim 1.

19. A method of treating obesity comprising the step of administering a therapeutically effective amount of a compound according to claim 1.

20. A pharmaceutical composition comprising a therapeutically effective amount of a compound of according to claim 1 in combination with a pharmaceutically suitable carrier.
